(19) United States
(12) Patent Application Publication
(10) Pub. No.: US 2026/0193351 A1
(43) Pub. Date: Jul. 9, 2026

(54) METHODS FOR TREATING CANCER WITH IMMUNOTHERAPY

(71) Applicant: THE UNIVERSITY OF CHICAGO, Chicago, IL (US)

(72) Inventors: Ralph R. WEICHSELBAUM, Chicago, IL (US); Hua L. LIANG, Chicago, IL (US); Sean P. PITRODA, Chicago, IL (US)

(73) Assignee: THE UNIVERSITY OF CHICAGO, Chicago, IL (US)

(21) Appl. No.: 19/132,096

(22) PCT Filed: Nov. 27, 2023
(86) PCT No.: PCT/US2023/081165

§ 371 Date: May 22, 2025

Related U.S. Application Data
(60) Provisional application No. 63/428,071, filed on Nov. 27, 2022.
(60) Provisional application No. 63/428,391, filed on Nov. 28, 2022.

(51) Int. Cl. C07K 16/28; A61K 39/00; A61N 5/10; A61P 35/04; C12Q 1/6886

(52) U.S. Cl. CPC C07K 16/2818; A61N 5/10; A61P 35/04; C07K 16/2827; C12Q 1/6886; A61K 2039/505; C12Q 2600/156

(57) ABSTRACT

Aspects herein relate, at least in part, to determining a treatment plan for a cancer patient based on a measured aneuploidy score from cancer cells from the patient.

This application claims priority to and the benefit of U.S. Provisional Application No. 63/428,071, filed Nov. 27, 2022, and U.S. Provisional Application No. 63/428,391, filed Nov. 28, 2022, the contents of which are both incorporated into the present application by reference in their entirety.

This invention was made with government support under CA195075 awarded by the National Institutes of Health. The government has certain rights in the invention.

### BACKGROUND OF THE INVENTION

### I. Field of the Invention

This invention relates to the field of oncology, genomics, and medicine.

### II. Background

Immunotherapy has revolutionized the management and treatment of patients with advanced cancers, yet most patients fail to respond to immune checkpoint inhibitors (ICIs). The landscape of biomarkers predicting response to immunotherapy has expanded, encompassing features including PD-L1 expression, signatures of CD8+ T cell function, tumor neoantigen load and TMB¹⁻⁵. TMB has been validated as a pan-cancer prognostic and predictive biomarker both in the setting of ICI treatment and in cancer at large in multiple independent studies^{1,6,7}. Recently, Samstein et al.¹ demonstrated in the largest immunogenomic data set of tumors treated with immunotherapy that higher nonsynonymous somatic TMB, defined as the top 20% within each cancer type, was associated with improved overall survival. The following year, the US Food and Drug Administration (FDA) issued pan-cancer approval of pembrolizumab for patients with a high TMB tumor, defined as ten or more mutations per megabase⁷. Although debate remains regarding the optimal threshold for defining high TMB tumors⁷, it is clear from prospective clinical trials that TMB provides important prognostic information regarding immunotherapy response⁴. However, regardless of the threshold utilized to define high TMB, the majority of patients exhibit a low TMB, and little is known regarding genomic predictors of outcome in this population. Thus, new biomarkers are needed to improve risk stratification of cancer immunotherapy.

### SUMMARY OF THE INVENTION

In general, the current disclosure relates to the discovery that aneuploidy levels in a tumor can predict the effectiveness of certain cancer therapies. Additionally, the disclosure shows, in certain aspects, that different tumors may have different levels of aneuploidy that are predictive of the effectiveness.

Accordingly, disclosed are methods of treating a cancer in a patient, methods of reducing metastasis in a patient, methods of improving the effectiveness of radiotherapy for treating cancer in a patient, methods of improving the effectiveness of an immune checkpoint blockade (ICB) therapy, and/or methods of improving the effectiveness of an ICB therapy in combination with a radiotherapy. The methods can comprise 1, 2, 3, 4, 5, 6, or more steps including any of the following: administering to the patient radiotherapy, administering to the patient an ICB therapy, administering to the patient radiotherapy and an ICB therapy, determining an aneuploidy score in a sample from the patient, determining tumor mutational burden, comparing an aneuploidy score from the patient to a reference score, and altering a treatment provided to the patient based on a measured aneuploidy score. In certain aspects, one or more of the preceding steps is specifically excluded.

In certain aspects, the radiotherapy and/or ICB therapy are administered after the cancer is determined to have a high aneuploidy score. Aneuploidy can be defined as an unbalanced number of chromosomes or chromosome arms. In certain aspects, an aneuploidy score is determined by the fraction of evaluable arms afflicted by arm-level somatic copy-number alterations. In certain aspects, an aneuploidy score is determined by measuring an amount of copy-number alterations in a cell, such as a cancer cell, from the patient. In certain aspects, an aneuploidy score is determined by measuring an amount of copy-number alterations in a population of cells, such as a population of cancer cells, from the patient. Aneuploidy can be measured by any method known in the art. In certain aspects, aneuploidy is measured by a copy number alteration assay. In certain aspects, aneuploidy is measured by sequencing one or more cells, such as one or more cancer cells, taken from a sample from the patient. In certain aspects, the aneuploidy score is measured by arm-level somatic copy number alterations.

In certain aspects, the aneuploidy score is measured by Arm-level Somatic Copy-number Events in Targeted Sequencing (ASCETS). In certain aspects, the cancer is determined to have a high aneuploidy score via biopsy and/or tumor resection. In certain aspects, the aneuploidy score is measured after biopsy and/or tumor resection by measuring aneuploidy in the biopsy and/or measuring aneuploidy in one or more tumor cells in the resected tumor. The biopsy and/or resected tumor can be processed (such as by fixation, cell dissociation, or other methods) to prepare the biopsy and/or resected tumor for an aneuploidy assay. The biopsy may be any biopsy including but not limited to needle biopsies, image-guided biopsy, surgical (excisional) biopsy, shave biopsy/punch biopsy, endoscopic biopsy, laparoscopic biopsy, bone marrow aspiration and biopsy, liquid biopsy or a combination thereof from tissue and/or tumors obtained from the patient. In certain aspects, the cancer is determined to have a high aneuploidy score by sequencing.

In certain aspects, a high aneuploidy score comprises an aneuploidy score greater than a reference score. In certain aspects, the reference score comprises an average aneuploidy score of a cohort of individuals. In certain aspects, the cohort of individuals comprises individuals known to or diagnosed to have cancer of the same type as the cancer in the patient.

In certain aspects, a patient is administered a specific therapy, such as radiotherapy and/or an ICB, after an aneuploidy score, such as an aneuploidy score from a population of cancer cells, is measured in the patient. In certain aspects, a patient is administered a specific therapy, such as radiotherapy and/or an ICB, after an aneuploidy score, such as an aneuploidy score from a population of cancer cells, is determined to be high. In certain aspects, a patient is administered a specific therapy, such as radiotherapy and/or an ICB, after an aneuploidy score, such as an aneuploidy score from a population of cancer cells, is determined to be higher than a reference score. In certain aspects, the reference score is a median aneuploidy score of table 3. In certain aspects, the reference score is at least, at most, or approximately 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, or any range derivable therein. In certain aspects, a high aneuploidy score is an aneuploidy score above 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, or any range derivable therein. In certain aspects, a high aneuploidy score is a score above the 50th, 51st, 52nd, 53rd, 54th, 55th, 56th, 57th, 58th, 59th, 60th, 61st, 62nd, 63rd, 64th, 65th, 66th, 67th, 68th, 69th, 70th, 71st, 72nd, 73rd, 74th, 75th, 76th, 77th, 78th, 79th, 80th, 81st, 82nd, 83rd, 84th, 85th, 86th, 87th, 88th, 89th, 90th percentile, or any range derivable therein, aneuploidy score of a cohort of individuals. In certain aspects, the cohort of individuals comprises individuals known to or diagnosed to have cancer of the same type as the cancer in the patient. For example, in certain aspects for treating a patient having non small-cell lung cancer, the reference score can be the 50th percentile (or any other value or percentile described herein) aneuploidy score in a cohort of individuals having, or diagnosed with having, non small-cell lung cancer.

The patient can have, be diagnosed with having, known to have, or be suspected of having a cancer. In certain aspects, the cancer has an indication for radiotherapy administration. In certain aspects, the cancer has an indication for ICB administration. In certain aspects, the cancer has an indication for ICB and radiotherapy administration. In certain aspects, the cancer originated in an organ of the individual selected from the group consisting of bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, uterus, and a combination thereof. In certain aspects, the cancer is a Stage I cancer, a Stage II cancer, a Stage III cancer, or a Stage IV cancer. The stage of the cancer can be determined by a clinician, such as a pathologist, or one skilled in the art. In certain aspects, the cancer is a not a glioma. In certain aspects, the cancer comprises a cancer derived from endoderm tissue. In certain aspects, the cancer comprises a non small-cell lung cancer or a large cell carcinoma. In certain aspects, the cancer comprises a myeloma or a melanoma. In certain aspects, the cancer comprises an immunologically cold tumor. In certain aspects, the cancer is metastatic. In certain aspects, the cancer is at risk of being metastatic. In certain aspects, the cancer is in multiple locations in the patient.

Certain aspects relate to administering a therapy, such as an ICB therapy. In certain aspects, the ICB comprises an anti-PD-1 agent, an anti-PD-L1 agent, and/or an anti-CTLA-4 agent. In certain aspects, the anti-PD-1 agent, an anti-PD-L1 agent, and/or an anti-CTLA-4 agent comprise an antibody, a small molecule, a biologic, an antisense oligonucleotide, and/or an RNAi molecule. In certain aspects, the ICB comprises ipilimumab, nivolumab, pembrolizumab, atezolizumab, avelumab, durvalumab, cemiplimab, or spartalizumab, or any combination thereof.

In certain aspects, the patient is administered radiotherapy and ICB therapy. In certain aspects, the patient is administered ICB therapy. In certain aspects, the patient is administered radiotherapy. In certain aspects, the patient receives radiotherapy while undergoing ICB therapy. In certain aspects, the radiotherapy and ICB therapy are administered sequentially. In certain aspects, 1, 2, 3, 4, 5, 6, 7, 8, 9, 10, 11, 12, 13, or 14, or any range derivable therein, days are between the radiotherapy and ICB therapy. In certain aspects, the radiotherapy is administered 1, 2, 3, 4, 5, 6, 7, or any range derivable therein, days and/or 1, 2, 3, or 4, or any range derivable therein, weeks prior to administering the ICB therapy. In certain aspects, the radiotherapy is administered 1, 2, 3, 4, 5, 6, 7, or any range derivable therein, days and/or 1, 2, 3, or 4, or any range derivable therein, weeks after administering the ICB therapy. In certain aspects, the radiotherapy is administered concurrently with the ICB therapy. In certain aspects, the radiotherapy is administered 1, 2, 3, 4, 5, or more times (or any range derivable therein) during a an ICB treatment regimen. It is also specifically contemplated that in certain aspects, the method excludes radiotherapy administered 2, 3, 4 weeks, 1, 2, 3, 4, 5, 6, 7, 8, 9, 10, 11, 12 months, or 1, 2, 3, years, or more (or any range derivable therein) before or after the administration of the ICB therapy.

Certain aspects relate to tumor mutational burden. In certain aspects, tumor mutational burden is measured in a sample from the patient. In certain aspects, tumor mutational burden is calculated in a population of cells, including a population of cancer cells, from the patient. In certain aspects, the tumor mutation burden is compared to a reference. In certain aspects, the reference comprises a measured or determined tumor mutational burden in a cohort of individuals. In certain aspects, the cohort of individuals comprises individuals known to or diagnosed to have cancer of the same type as the cancer in the patient. In certain aspects, a high tumor mutational burden is a tumor mutational burden between the 50^{th}-100^{th} percentile. In certain aspects, a high tumor mutational burden is a tumor mutational burden above the 50th, 51st, 52nd, 53rd, 54th, 55th, 56th, 57th, 58th, 59th, 60th, 61st, 62nd, 63rd, 64th, 65th, 66th, 67th, 68th, 69th, 70th, 71st, 72nd, 73rd, 74th, 75th, 76th, 77th, 78th, 79th, 80th, 81st, 82nd, 83rd, 84th, 85th, 86th, 87th, 88th, 89th, 90th percentile, or any range derivable therein, tumor mutational burden of a cohort of individuals. In certain aspects, a high tumor mutational burden is a tumor mutational burden between the 0-49^{th} percentile. In certain aspects, a low tumor mutational burden is a tumor mutational burden below the 50th, 51st, 52nd, 53rd, 54th, 55th, 56th, 57th, 58th, 59th, 60th, 61st, 62nd, 63rd, 64th, 65th, 66th, 67th, 68th, 69th, 70th, 71st, 72nd, 73rd, 74th, 75th, 76th, 77th, 78th, 79th, 80th, 81st, 82nd, 83rd, 84th, 85th, 86th, 87th, 88th, 89th, 90th percentile, or any range derivable therein, tumor mutational burden of a cohort of individuals.

Also disclosed are methods for determining the effectiveness of a radiotherapy and/or immune checkpoint blockade (ICB) therapy in treating a cancer in an individual, methods for determining the effectiveness of an ICB therapy in treating a cancer in an individual, methods for determining the effectiveness of a radiotherapy in treating a cancer in an individual, methods of prognosing a patient indicated for radiotherapy and/or ICB therapy, and methods of evaluating an aneuploidy score of a cancer in a patient. The methods can comprise 1, 2, 3, 4, 5, 6, 7, 8 or more steps including any of the following: determining an aneuploidy score of the cancer, calculating an aneuploidy score, measuring aneuploidy in a biological sample, comparing an aneuploidy score to a reference score, determining a likelihood of effectiveness of a cancer therapy based on the aneuploidy score relative to a reference score, measuring a tumor mutational burden, calculating a tumor mutational burden, determining a tumor mutational burden, and administering a cancer therapy. In certain aspects, one or more of the preceding steps is specifically excluded.

The cancer can be any cancer, including any cancer described herein. The ICB therapy can be any ICB therapy, including any ICB described herein. The aneuploidy score can be measured, determined, and/or calculated using any method described herein.

Also disclosed are methods of treating cancer in a patient that has received radiotherapy. In certain aspects, the methods comprise one or more steps including administering an immune checkpoint blockade (ICB) therapy if the patient is determined to have an increase in at least one immune checkpoint gene product after receiving the radiotherapy. The cancer can be any cancer, including any cancer described herein.

In certain aspects, the ICB therapy comprises an anti-PD-1 agent, an anti-PD-L1 agent, and/or an anti-CTLA-4 agent. In certain aspects, the anti-PD-1 agent, an anti-PD-L1 agent, and/or an anti-CTLA-4 agent comprise an antibody, a small molecule, a biologic, an antisense oligonucleotide, and/or an RNAi molecule. In certain aspects, the ICB therapy comprises ipilimumab, nivolumab, pembrolizumab, atezolizumab, avelumab, durvalumab, cemiplimab, or spartalizumab, or any combination thereof. In certain aspects, the immune checkpoint gene product comprises mRNA and/or protein produced from a PD-1, PD-L1, and/or CTLA-4 gene. In certain aspects, the increase is determined relative to an amount of immune checkpoint gene product measured in the patient prior to the patient receiving the radiotherapy. In certain aspects, the increase is determined relative to a standard level of the immune checkpoint gene product in one or more healthy individuals.

In certain aspects, the immune checkpoint gene product is measured in a biopsy. In certain aspects, the immune checkpoint gene product is measured in circulating immune cells. In certain aspects, the cancer has an indication for radiotherapy administration.

Certain aspects are related to methods of treating cancer in a patient, the method comprising administering to the patient a second therapy after the cancer is measured for an aneuploidy score and/or tumor mutational burden, wherein the patient has received a first therapy and wherein the second therapy comprises radiotherapy and/or an immune checkpoint blockade (ICB) therapy. In some aspects, the aneuploidy score and/or tumor mutational burden is measured before the patient has received the first therapy. In some aspects, the aneuploidy score and/or tumor mutational burden is measured during the patient receiving the first therapy. In some aspects, the aneuploidy score and/or tumor mutational burden is measured after the patient has received the first therapy. In certain aspects, the aneuploidy score is a high aneuploidy score. In certain aspects, the second therapy comprises radiotherapy and an immune checkpoint blockade (ICB) when the aneuploidy score is a high aneuploidy score. In certain aspects, the aneuploidy score is a low aneuploidy score. In certain aspects, the second therapy comprises an immune checkpoint blockade (ICB) when the aneuploidy score is a low aneuploidy score. In some aspects, the presence of high aneuploidy and lower TMB indicates that the cancer of the patient is poorly responsive to immunotherapy alone and should also receive radiotherapy. In some aspects, the presence of low aneuploidy and lower TMB indicates that the cancer of the patient is responsive to immunotherapy alone. In some aspects, the first therapy comprises an ICB. In some aspects, the first therapy comprises radiotherapy and an ICB. In some aspects, the first therapy comprises ICB and/or radiotherapy.

Also disclosed are methods of improving an immune checkpoint blockade therapy received by a patient, the method comprising administering a radiotherapy to the patient, wherein the patient has been determined to have a high aneuploidy score and low tumor mutational burden. Also disclosed are methods of improving an immune checkpoint blockade therapy received by a patient, the method comprising administering a radiotherapy to the patient, wherein the patient has been determined to have a high aneuploidy score and/or low tumor mutational burden.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more,” “at least one,” and “one or more than one.” Any term used in singular form also comprise plural form and vice versa.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” “(x and z) or y,” or “x or y or z.” It is specifically contemplated that x, y, or z may be specifically excluded from an aspect or aspect.

As used herein, “patient” can refer to a human or a human patient. In some aspects, “individual” is interchangeable with “patient”.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments and aspects described in the context of the term “comprising” may also be implemented in the context of the term “consisting of” or “consisting essentially of.”

As used herein, a “cold” tumor can be a tumor that has not been infiltrated with immune cells, such as T cells. Cold tumors include those described by Galon and Bruni, Nature Reviews Drug Discovery volume 18, pages 197-218 (2019).

It is contemplated that any aspect discussed in this specification can be implemented with respect to any method or composition of the invention, and vice versa. Furthermore, compositions of the invention can be used to achieve methods of the invention.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Use of the one or more sequences or compositions may be employed based on any of the methods described herein. Other aspects and embodiments are discussed throughout this application. Any embodiment or aspect discussed with respect to one aspect of the disclosure applies to other aspects of the disclosure as well and vice versa.

It is specifically contemplated that any limitation discussed with respect to one embodiment or aspect of the invention may apply to any other embodiment or aspect of the invention. Furthermore, any composition of the invention may be used in any method of the invention, and any method of the invention may be used to produce or to utilize any composition of the invention. Aspects of an embodiment set forth in the Examples are also aspects that may be implemented in the context of aspects discussed elsewhere in a different Example or elsewhere in the application, such as in the Summary of the Invention, Brief Description of the Drawings, Detailed Description of the Invention, and/or Claims.

Other objects, features and advantages of the present invention will become apparent from the following detailed description. It should be understood, however, that the detailed description and the specific examples, while indicating specific aspects of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1G show aneuploidy and TMB are synergistic predictors of survival following immunotherapy. a, Distribution of aneuploidy scores across cancer types. Boxplot elements are defined in the Methods. b, Forest plot of the multivariable model of overall survival using TMB, aneuploidy score and clinical features. Wald test multivariable adjusted P values are displayed. c, Forest plot of overall survival within each cancer type using TMB and aneuploidy score in a multivariable model with ICI drug class; n=1,660 biologically independent samples. d, Spearman correlation between TMB and aneuploidy score across all samples (n=1,660). e, Spearman correlation between aneuploidy score and FGA across all samples (n=1,660). f, Comparison of multivariable hazard ratios using aneuploidy score. and FGA in a multivariable Cox proportional hazards model with TMB and drug class; paired Wilcoxon test; n=1,660 biologically independent samples. For cancer types where the hazard ratio was less than 1 (glioma and esophagogastric cancer), the inverse of the hazard ratio is displayed. g, Association of specific chromosome arm aneuploidies with overall survival in a multivariable Cox proportional hazards model with TMB and drug class. The left panel shows copy-number gains and the right panel shows losses. The dotted line represents false discovery rate-corrected P=0.05. Forest and boxplot elements for all panels are described in the Methods. The color legend displayed adjacent to panel g applies to panels e-g.

FIGS. 2A-2D show defining a clinically applicable threshold of high aneuploidy. a, Box plots of candidate thresholds to split patients into high versus low aneuploidy score at each threshold. 1,660 multivariable Cox models as part of the leave-one-out cross validation analysis were constructed with aneuploidy score binned at the candidate threshold, drug class and TMB binned at the highest 20th percentile. The Wald P value and multivariable hazard ratio for aneuploidy score are displayed. Boxplot elements are defined in the Methods. b, Forest plot of overall survival within each cancer type using TMB binned at the highest 20^{th} percentile and aneuploidy score binned at the 50th percentile in a multivariable model with ICI drug class; n=1,660 biologically independent samples. c, Kaplan-Meier analysis of TMB binned at the highest 20th percentile and aneuploidy score binned at the 50th percentile. P values next to the legend indicate pairwise comparisons within each row. Global P value is displayed in the bottom left of the plot. AS, aneuploidy score; H, high; L, low. d, Comparison of 2-year overall survival stratified by cancer type for high versus low TMB (binned at the highest 20th percentile) (left) and high versus low aneuploidy score (binned at 50th percentile) among low TMB tumors; Wilcoxon test; n=1,660 biologically independent samples. Forest and boxplot elements for all panels are defined in the Methods.

FIGS. 3A-3B shows a schematic of clinical trial. a, Randomized phase I COSINR clinical trial design. b, Schematic demonstrating the differences in biopsy time points by treatment arm and their implications for interpretation of clinical, genomic and transcriptomic results. IO, immunotherapy.

FIGS. 4A-4C show therapy-induced changes in the genomic landscape. a, Clonal evolution of somatic mutations during treatment. Each box corresponds to an individual patient (n=18 patients). Axes indicate the fraction of cancer nuclei (CCF) harboring the mutation; oncogenic mutations are highlighted in blue. All mutations with a calculable CCF (that is, FACETS purity>0 and copy number available for the mutation locus) are plotted. b, Differences in on-treatment density of tumor (TTF1+/CK5+) cells as determined by multiplexed immunofluorescence between tumors in which no genomic variants were detected on therapy by whole exome sequencing (eliminated; patients 12, 13, 14, 32 and 49) and tumors with detected on-treatment variants (persistent) (two-sided unpaired Wilcoxon test). Multiplexed immunofluorescence data are not available for patients 15 and 47 (SBRT, n=6 patients; SBRT+ipi/nivo, n=6 patients). The top and bottom edges represent the first and third quartiles, respectively. The center line represents the median. Whiskers extend to the furthest data points before any outliers (within 1.5× the interquartile range). Outliers are plotted as points above and below the box-and-whisker plot. c, Changes in aneuploidy score and TMB following treatment (two-sided paired Wilcoxon test; SBRT, n=10 patients; SBRT+ipi/nivo, n=8 patients). Box plot elements are the same as in part b.

FIGS. 5A-5B show therapy-induced changes in the tumor transcriptome. a, ssGSEA hallmark pathway changes during treatment in paired samples (n=15 patients); two-sided Wilcoxon test for median changes (on-treatment (On-tx)-pretreatment (Pre-tx)) between treatment arms. Significant pathways are highlighted in blue (P<0.05). b, Heatmap of fold changes (log₂(on-treatment FPKM/pretreatment FPKM)) in gene expression of manually selected immune genes found to be significantly upregulated or downregulated following SBRT and/or SBRT+ipi/nivo (FDR<0.1). Orange, upregulation; blue, downregulation. Upward and downward arrows reflect upregulation or downregulation of the genes in that cluster, respectively; dashed lines indicate no change. Columns represent patients (SBRT, n=8 patients; SBRT+ipi/nivo, n=7 patients). c, Changes in ESTIMATE and xCell immune scores in each treatment arm (two-sided paired Wilcoxon test; SBRT, n=8 patients; SBRT+ipi/nivo, n=7 patients). Box plot elements are the same as in FIG. 4B.

FIGS. 6A-6E show immunological evolution during treatment. a, Differential changes in log₂(fold changes) in xCell immune cell-associated signatures following SBRT and SBRT+ipi/nivo. All T cell-associated signatures and any signature with P<0.05 are labeled. Yellow points indicate P<0.05. Two-sided Wilcoxon test comparing median log₂(fold change) of on-treatment versus pretreatment cell populations between the SBRT+ipi/nivo and SBRT groups (SBRT, n=8 patients; SBRT+ipi/nivo, n=7 patients). Significant (yellow) points are differentially changed cell populations following treatment, comparing the two treatment arms. b, Evolution of TCR clonotypes during treatment in paired samples (two-sided Fisher's exact test; SBRT, n=8 patients; SBRT+ipi/nivo, n=7 patients). c, Change (on-treatment score−pretreatment score) in effector T cell IFNγ-associated gene signature (two-sided paired Wilcoxon test; SBRT, n=8 patients; SBRT+ipi/nivo, n=7 patients). Box plot elements are the same as in FIG. 4B. d, Correlation between IFNγ signature and CD8⁺ T cell density as determined by multiplexed immunofluorescence in pretreatment and on-treatment samples (two-sided Spearman correlation; SBRT, n=6 patients; SBRT+ipi/nivo, n=6 patients). e, Selected multiplexed immunofluorescence images from one tumor in each treatment arm. Scale bars, 50 μm (×200).

FIGS. 7A-7C show evaluation of immunotherapy biomarkers. a, Forest plot demonstrating univariable hazard ratios for PFS and OS of established pretreatment biomarkers of immunotherapy response and aneuploidy score; PD-L1 expression (high (≥50%) versus low (<50%) tumor proportion score (TPS)), n=34 patients; TMB and aneuploidy score (continuous variables), n=22 patients; neoantigens (continuous variable), n=18 patients; and T cell IFNγ score (continuous variable), n=15 patients. Center indicates hazard ratio point estimate, and lines indicate confidence intervals; confidence intervals exceeding limits of the x axis were truncated. b, Two-sided Spearman correlation between pretreatment aneuploidy score and change (on-treatment-pretreatment) in tumor purity (SBRT, n=10 patients; SBRT+ipi/nivo, n=8 patients). Patient identification numbers are labeled, and those highlighted in orange are presented in part c. c, Representative images of pretreatment and on-treatment samples from high-aneuploid tumors treated with SBRT and SBRT+ipi/nivo (hematoxylin and eosin staining; scale bars, 50 μm (×200)). Both pretreatment samples show adenocarcinoma. The on-treatment sample from patient 13 predominantly shows necrosis with small areas of residual tumor compared with that from patient 28, which shows extensive residual adenocarcinoma alongside small regions of necrosis. Dotted orange lines outline viable tumor.

FIGS. 8A-8H show aneuploidy as a biomarker of combination radiotherapy and ICB response. a, Two-sided Spearman correlation between pretreatment aneuploidy score and ssGSEA hallmark pathways (n=15 patients). Pathways highlighted in green are significant (two-sided P<0.05). b, Correlation between pretreatment aneuploidy score, and ESTIMATE and xCell immune scores; two-sided Spearman correlation (n=15 patients). c, Comparison of aneuploidy score by PD-L1 expression in the combined UC and COSINR cohorts (high PD-L1, n=52 patients; low PD-L1, n=83 patients); high PD-L1 is defined as ≥50% TPS expression (two-sided Wilcoxon test). Box plot elements are the same as in FIG. 4B. d, Association between high aneuploidy score (≥median) and low aneuploidy score (<median) and OS by treatment arm (log-rank test). e, Comparison of disease control rate (defined as lack of disease progression) in nonirradiated lesions by treatment arm and aneuploidy score (two-sided Fisher's exact test; for the concurrent (Conc.) arm, n=5 patients for low aneuploidy score and n=5 patients for high aneuploidy score; and for the sequential (Seq.) arm, n=6 patients for low aneuploidy score and n=6 patients for high aneuploidy score). AS, aneuploidy score. f, Comparison of patterns of failure in nonirradiated lesions by treatment arm and aneuploidy score (two-sided Fisher's exact test). Yellow indicates the appearance of new lesions, and green indicates progression in an existing, unirradiated site. Sample sizes are defined in e. g, Association of treatment modalities with survival in high aneuploidy score (≥median) and low aneuploidy score (<median) tumors in the UC cohort (log-rank test). Dotted lines represent subdivisions of the radiotherapy (RT)+ICB treatment group into patients treated with concurrent (maroon) or sequential (yellow) RT+ICB. h, Synergistic prediction of survival by TMB and aneuploidy score (tumors split by the cross-validated threshold identified in the UC cohort, >0.42 (high) versus<0.42 (low)) in an independent metastatic NSCLC cohort treated with immunotherapy. Orange lines, patients with high aneuploidy score (≥0.42); gray lines, patients with low aneuploidy score (<0.42) (two-sided log-rank test). CR, complete response; PD, progressive disease; PR, partial response; SD, stable disease.

FIGS. 9A-9B shows consort diagram of patient selection and data analytical framework. a) Patient selection for clinical and genomic analyses. Patients were excluded based on manual pathologic review and inspection of genomic results. (b) Schematic of genomic and transcriptomic analysis workflow.

FIG. 10 shows OncoPrint of COSINR patient cohort. OncoPrint plot of tumors which successfully underwent whole exome sequencing (n=40 samples). Paired samples are adjacent with individual patients separated by larger white breaks. Clinical and pathological data are displayed above variants. Dominant mutational processes reflect the mutational signature etiology with the largest contribution to the sample's overall mutational profile. Percentages reflect the prevalence of gene alterations in the pre-treatment samples. Bar graphs on the right of the plot reflect the total number of gene alterations across all samples.

FIGS. 11A-11C show baseline clinical characteristics of COSINR cohort. (a) Association of clinicopathologic variables with progression-free (top) and overall survival (bottom). (b) Progression-free and overall survival of entire cohort by treatment arm. Dotted vertical lines represent median survival; two-sided Log rank test. (c) Progression-free and overall survival by treatment arm in the subset of patients used for molecular analysis; two-sided Log-rank test; dotted lines represent median survival (n=22 patients).

FIGS. 12A-12F show changes in genomic and transcriptomic features on therapy. (a) Clonal evolution of somatic mutations on treatment. Each box corresponds to an individual patient. Axes indicate the variant allele fractions (VAFs) of each mutation (x-axis: pre-treatment; y-axis: on-treatment); oncogenic mutations highlighted in blue; (n=18 patients). (b) Differences in on-treatment density of (TTF1+/CK5+) tumor cells as determined by mIF between treatment arms; (SBRT n=6 patients, SBRT+Ipi/Nivo n=6 patients); two-sided Wilcoxon test. The top and bottom edges represent the 1^{st} and 3^{rd} quartiles, respectively; the center line represents the median; whiskers extend to the farthest data points which do not represent outliers (within 1.5× the interquartile range); outliers are plotted as points above and below the box-and-whisker plot. (c) On treatment changes in purity and ploidy; two-sided paired Wilcoxon test. Patient #12 was excluded from the SBRT+ipi/nivo group because the tumor purity could not accurately be determined for the on-treatment sample (SBRT+Ipi/Nivo n=7 patients, SBRT n=10 patients). Boxplot elements are defined in the legend of panel b. (d) Changes in ssGSEA Hallmark pathway scores on-treatment (SBRT n=8 patients, SBRT+Ipi/Nivo n=7 patients); two-sided paired Wilcoxon P<0.05 are highlighted in blue. (e) Plots illustrating patient-level changes in ssGSEA Hallmark pathways determined to be significantly differentially changed between treatment arms (see FIG. 4D); (SBRT n=8 patients, SBRT+Ipi/Nivo n=7 patients); two-sided paired Wilcoxon test. Box plot elements are defined in the legend of panel b. (f) Changes in ESTIMATE stromal score on treatment in each treatment arm; (SBRT n=8 patients, SBRT+Ipi/Nivo n=7 patients); two-sided paired Wilcoxon test. Box plot elements are defined in the legend of panel b.

FIGS. 13A-13E show changes in T cell landscape during treatment. (a) Balance in baseline immune cell signatures across COSINR treatment arms using the four xCell signature matrices. Dashed line indicates SBRT=SBRT+Ipi/Nivo. All T-cell associated signatures and any signature with two-sided Wilcoxon P<0.05 are labeled. Blue points are P<0.05 (SBRT n=8 patients, SBRT+Ipi/Nivo n=7 patients); paired two-sided Wilcoxon signed-rank test. (b) Changes in CD8+ T cell populations using the 4 xCell signatures; (SBRT n=8 patients, SBRT+Ipi/Nivo n=7 patients). Box plot elements are defined in the legend of FIG. 12B (c) Changes in TCR richness and evenness (SBRT n=8 patients, SBRT+Ipi/Nivo n=7 patients); two-sided paired Wilcoxon test. Box plot elements are defined in the legend of FIG. 12B. (d) Evolution of TCR clonotypes at a per-patient level; horizontal dotted lines represent the median number of novel TCRs per treatment group (SBRT n=8 patients, SBRT+Ipi/Nivo n=7 patients). (e) Two-sided Spearman correlation between pre-treatment (left) and on-treatment (right) CD8+ T cell populations and the number (richness) of TCRs (n=15 patients).

FIGS. 14A-14E show integrative T cell characterization using RNA-seq and immunofluorescence. (a) Correlation between xCell immune cell type scores and CD8+ T cell density as determined by mIF; two-sided Spearman correlation (SBRT n=8 patients, SBRT+Ipi/Nivo n=7 patients). (b) Correlation between change in effector T cell IFNγ signature and change in CD8+ T cell density; two-sided Spearman correlation (SBRT n=8 patients, SBRT+Ipi/Nivo n=7 patients). (c) Changes in CD8+ T cell density as determined by mIF by treatment arm; (SBRT n=6 patients, SBRT+Ipi/Nivo n=6 patients); two-sided Wilcoxon test. Box plot elements are defined in the legend of FIG. 12B. (d) Change in naïve T cell gene expression signature by treatment arm; (SBRT n=8 patients, SBRT+Ipi/Nivo n=7 patients); two-sided Wilcoxon test. Box plot elements are defined in FIG. 12B. (e) Change in the fraction of PD-L1-positive tumor and stromal cells by mIF across treatment arms; (SBRT n=6 patients, SBRT+Ipi/Nivo n=6 patients); two-sided Wilcoxon test. Box plot elements are defined in the legend of FIG. 12B.

FIGS. 15A-15E show association between immunotherapy biomarkers and survival. Association of pre-treatment (a) effector T cell IFNγ signature (n=15 patients), (b) TMB (n=22 patients), (c) PD-L1 expression (n=34 patients), (d) neoantigen count (n=18 patients), and (e) aneuploidy score (n=22 patients) with progression-free (left) and overall survival (right). Variables were split at the median; two-sided Log-rank test.

FIGS. 16A-16H show aneuploidy biomarker development in mNSCLC (a) PFS for COSINR patients with high aneuploidy score (AS, ≥median) (left) and low AS (<median, right) tumors; two-sided Log-rank test. (b) Scatter plot of AS and tumor purity (COSINR, n=22 patients). (c) Comparison of number of pre-treatment organ sites by COSINR treatment arm and aneuploidy group (n=22 patients); two-sided Wilcoxon test. Box plot elements are defined in the legend of FIG. 12B. (d) Association of clinical and pathological factors with overall survival in UC cohort (n=58 patients). Variables tested were age, sex (M vs. F), presence of brain or liver metastases, smoking status (ever vs. never), PD-L1 expression (≥50% vs.<50%), histology (adenocarcinoma vs. other), number of disease sites, TMB, ECOG (0-1 vs. 2-3), and ICB paradigm (monotherapy vs. combination therapy). Variables significantly associated with OS are highlighted in blue; two-sided Wald test. (e) Distribution of AS in COSINR (n=22 patients), UC (n=58 patients), and TCGA (n=500 patients) cohorts; dotted line represents high AS threshold (0.42). (f) Selection of optimal high AS threshold based on leave-one-out cross validation analysis; bars: 95% confidence interval; points: mean. Grey lines outline optimal AS threshold (0.42) (n=58 patients). (g) Differences in OS in high AS (≥0.42) and low AS (<0.42) groups in UC validation cohort using the derived optimal threshold; two-sided Log-rank test. Dotted maroon and yellow lines represent subdivisions of the RT/ICB treatment group into patients treated with concurrent (maroon) or sequential (yellow) RT+ICB. (h) Application of the derived optimal threshold (0.42) to the COSINR cohort (OS); two-sided Log-rank test.

### DETAILED DESCRIPTION OF THE INVENTION

### I. Detecting a Genetic Signature

Particular aspects concern the methods of detecting a genetic signature in an individual. In certain aspects, the genetic signature comprises an aneuploidy score. In certain aspects, the genetic signature comprises one or more chromosomal abnormalities. In certain aspects, the genetic signature comprises one or more copy number alterations.

In certain aspects, detecting the genetic signature comprises detecting abnormalities on one or more chromosomal arms. In certain aspects, detecting the genetic signature comprises detecting aneuploidy in a sample from a patient. In certain aspects, detecting the genetic signature comprises detecting aneuploidy in cancer cells from a patient. In certain aspects, detecting the genetic signature comprises detecting arm-level somatic copy-number changes, including by Arm-level Somatic Copy-number Events in Targeted Sequencing (ASCETS).

In some aspects, the method for detecting the genetic signature may include selective oligonucleotide probes, arrays, allele-specific hybridization, molecular beacons, restriction fragment length polymorphism analysis, enzymatic chain reaction, flap endonuclease analysis, primer extension, 5′-nuclease analysis, oligonucleotide ligation assay, single strand conformation polymorphism analysis, temperature gradient gel electrophoresis, denaturing high performance liquid chromatography, high-resolution melting, DNA mismatch binding protein analysis, surveyor nuclease assay, sequencing, or a combination thereof, for example. The method for detecting the genetic signature may include fluorescent in situ hybridization, comparative genomic hybridization, arrays, polymerase chain reaction, sequencing, or a combination thereof, for example. The detection of the genetic signature may involve using a particular method to detect one feature of the genetic signature and additionally use the same method or a different method to detect a different feature of the genetic signature. Multiple different methods independently or in combination may be used to detect the same feature or a plurality of features.

### A. Single Nucleotide Polymorphism (SNP) Detection

Particular aspects of the disclosure concern methods of detecting a SNP in an individual. The methods of detecting SNPs may be used to determine tumor mutational burden. In certain aspects, tumor mutational burden comprises the number of nonsynonymous mutations in each sample divided by a reference such as a total genome size and/or a sequencing bait size.

One may employ any of the known general methods for detecting SNPs for detecting the particular SNP in this disclosure, for example. Such methods include, but are not limited to, selective oligonucleotide probes, arrays, allele-specific hybridization, molecular beacons, restriction fragment length polymorphism analysis, enzymatic chain reaction, flap endonuclease analysis, primer extension, 5′-nuclease analysis, oligonucleotide ligation assay, single strand conformation polymorphism analysis, temperature gradient gel electrophoresis, denaturing high performance liquid chromatography, high-resolution melting, DNA mismatch binding protein analysis, surveyor nuclease assay, sequencing, or a combination thereof.

In some aspects of the disclosure, the method used to detect the SNP comprises sequencing nucleic acid material from the individual and/or using selective oligonucleotide probes. Sequencing the nucleic acid material from the individual may involve obtaining the nucleic acid material from the individual in the form of genomic DNA, complementary DNA that is reverse transcribed from RNA, or RNA, for example. Any standard sequencing technique may be employed, including Sanger sequencing, chain extension sequencing, Maxam-Gilbert sequencing, shotgun sequencing, bridge PCR sequencing, high-throughput methods for sequencing, next generation sequencing, RNA sequencing, or a combination thereof. After sequencing the nucleic acid from the individual, one may utilize any data processing software or technique to determine which particular nucleotide is present in the individual at the particular SNP.

In some aspects, the nucleotide at the particular SNP is detected by selective oligonucleotide probes. The probes may be used on nucleic acid material from the individual, including genomic DNA, complementary DNA that is reverse transcribed from RNA, or RNA, for example. Selective oligonucleotide probes preferentially bind to a complementary strand based on the particular nucleotide present at the SNP. For example, one selective oligonucleotide probe binds to a complementary strand that has an A nucleotide at the SNP on the coding strand but not a G nucleotide at the SNP on the coding strand, while a different selective oligonucleotide probe binds to a complementary strand that has a G nucleotide at the SNP on the coding strand but not an A nucleotide at the SNP on the coding strand. Similar methods could be used to design a probe that selectively binds to the coding strand that has a C or a T nucleotide, but not both, at the SNP. Thus, any method to determine binding of one selective oligonucleotide probe over another selective oligonucleotide probe could be used to determine the nucleotide present at the SNP.

One method for detecting SNPs using oligonucleotide probes comprises the steps of analyzing the quality and measuring quantity of the nucleic acid material by a spectrophotometer and/or a gel electrophoresis assay; processing the nucleic acid material into a reaction mixture with at least one selective oligonucleotide probe, PCR primers, and a mixture with components needed to perform a quantitative PCR (qPCR), which could comprise a polymerase, deoxynucleotides, and a suitable buffer for the reaction; and cycling the processed reaction mixture while monitoring the reaction. In one aspect of the method, the polymerase used for the qPCR will encounter the selective oligonucleotide probe binding to the strand being amplified and, using endonuclease activity, degrade the selective oligonucleotide probe. The detection of the degraded probe determines if the probe was binding to the amplified strand.

Another method for determining binding of the selective oligonucleotide probe to a particular nucleotide comprises using the selective oligonucleotide probe as a PCR primer, wherein the selective oligonucleotide probe binds preferentially to a particular nucleotide at the SNP position. In some aspects, the probe is generally designed so the 3′ end of the probe pairs with the SNP. Thus, if the probe has the correct complementary base to pair with the particular nucleotide at the SNP, the probe will be extended during the amplification step of the PCR. For example, if there is a T nucleotide at the 3′ position of the probe and there is an A nucleotide at the SNP position, the probe will bind to the SNP and be extended during the amplification step of the PCR. However, if the same probe is used (with a T at the 3′ end) and there is a G nucleotide at the SNP position, the probe will not fully bind and will not be extended during the amplification step of the PCR.

In some aspects, the SNP position is not at the terminal end of the PCR primer, but rather located within the PCR primer. The PCR primer should be of sufficient length and homology in that the PCR primer can selectively bind to one variant, for example the SNP having an A nucleotide, but not bind to another variant, for example the SNP having a G nucleotide. The PCR primer may also be designed to selectively bind particularly to the SNP having a G nucleotide but not bind to a variant with an A, C, or T nucleotide. Similarly, PCR primers could be designed to bind to the SNP having a C or a T nucleotide, but not both, which then does not bind to a variant with a G, A, or T nucleotide or G, A, or C nucleotide respectively. In particular aspects, the PCR primer is at least or no more than 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or more nucleotides in length with 100% homology to the template sequence, with the potential exception of non-homology the SNP location. After several rounds of amplifications, if the PCR primers generate the expected band size, the SNP can be determined to have the A nucleotide and not the G nucleotide.

### B. Copy Number Alteration Detection

Particular aspects of the disclosure concern methods of detecting one or more copy number alterations (CNA), including CNAs of a particular allele. One can utilize any known method for detecting CNAs to detect the CNAs. Such methods include fluorescent in situ hybridization, comparative genomic hybridization, arrays, polymerase chain reaction, sequencing, or a combination thereof, for example. Array platforms such as those from Agilent, Illumina, or Affymetrix may be used, or custom arrays could be designed. One example of how an array may be used includes methods that comprise one or more of the steps of isolating nucleic acid material in a suitable manner from an individual suspected of having the CNA and, at least in some cases from an individual or reference genome that does not have the CNA; processing the nucleic acid material by fragmentation, labelling the nucleic acid with, for example, fluorescent labels, and purifying the fragmented and labeled nucleic acid material; hybridizing the nucleic acid material to the array for a sufficient time, such as for at least 24 hours; washing the array after hybridization; scanning the array using an array scanner; and analyzing the array using suitable software. The software may be used to compare the nucleic acid material from the individual suspected of having the CNA to the nucleic acid material of an individual who is known not to have the CNA or a reference genome.

In some aspects, detection of a CNA is achieved by polymerase chain reaction (PCR). PCR primers can be employed to amplify nucleic acid at or near the CNA wherein an individual with a CNA will result in measurable higher levels of PCR product when compared to a PCR product from a reference genome. The detection of PCR product amounts could be measured by quantitative PCR (qPCR) or could be measured by gel electrophoresis, as examples. Quantification using gel electrophoresis comprises subjecting the resulting PCR product, along with nucleic acid standards of known size, to an electrical current on an agarose gel and measuring the size and intensity of the resulting band. The size of the resulting band can be compared to the known standards to determine the size of the resulting band. In some aspects, the amplification of the CNA will result in a band that has a larger size than a band that is amplified, using the same primers as were used to detect the CNA, from a reference genome or an individual that does not have the CNA being detected. The resulting band from the CNA amplification may be nearly double, double, or more than double the resulting band from the reference genome or the resulting band from an individual that does not have the CNA being detected. In some aspects, the CNA can be detected using nucleic acid sequencing. Sequencing techniques that could be used include, but are not limited to, whole genome sequencing, whole exome sequencing, and/or targeted sequencing.

### C. DNA Sequencing

In some aspects, DNA may be analyzed by sequencing. The DNA may be prepared for sequencing by any method known in the art, such as library preparation, hybrid capture, sample quality control, product-utilized ligation-based library preparation, or a combination thereof. The DNA may be prepared for any sequencing technique. In some aspects, a unique genetic readout for each sample may be generated by genotyping one or more highly polymorphic SNPs. In some aspects, sequencing, such as 76 base pair, paired-end sequencing, may be performed to cover approximately 70%, 75%, 80%, 85%, 90%, 95%, 99%, or greater percentage of targets at more than 20×, 25×, 30×, 35×, 40×, 45×, 50×, or greater than 50× coverage. In certain aspects, mutations, SNPS, INDELS, copy number alterations (somatic and/or germline), or other genetic differences may be identified from the sequencing using at least one bioinformatics tool, including VarScan2, any R package (including CopywriteR) and/or Annovar.

### D. RNA Sequencing

In some aspects, RNA may be analyzed by sequencing. The RNA may be prepared for sequencing by any method known in the art, such as poly-A selection, cDNA synthesis, stranded or nonstranded library preparation, or a combination thereof. The RNA may be prepared for any type of RNA sequencing technique, including stranded specific RNA sequencing. In some aspects, sequencing may be performed to generate approximately 10M, 15M, 20M, 25M, 30M, 35M, 40M or more reads, including paired reads. The sequencing may be performed at a read length of approximately 50 bp, 55 bp, 60 bp, 65 bp, 70 bp, 75 bp, 80 bp, 85 bp, 90 bp, 95 bp, 100 bp, 105 bp, 110 bp, or longer. In some aspects, raw sequencing data may be converted to estimated read counts (RSEM), fragments per kilobase of transcript per million mapped reads (FPKM), and/or reads per kilobase of transcript per million mapped reads (RPKM). In some aspects, one or more bioinformatics tools may be used to infer stroma content, immune infiltration, and/or tumor immune cell profiles, such as by using upper quartile normalized RSEM data.

### E. Proteomics

In some aspects, protein may be analyzed by mass spectrometry. The protein may be prepared for mass spectrometry using any method known in the art. Protein, including any isolated protein encompassed herein, may be treated with DTT followed by iodoacetamide. The protein may be incubated with at least one peptidase, including an endopeptidase, proteinase, protease, or any enzyme that cleaves proteins. In some aspects, protein is incubated with the endopeptidase, LysC and/or trypsin. The protein may be incubated with one or more protein cleaving enzymes at any ratio, including a ratio of μg of enzyme to μg protein at approximately 1:1000, 1:100, 1:90, 1:80, 1:70, 1:60, 1:50, 1:40, 1:30, 1:20, 1:10, 1:1, or any range between. In some aspects, the cleaved proteins may be purified, such as by column purification. In certain aspects, purified peptides may be snap-frozen and/or dried, such as dried under vacuum. In some aspects, the purified peptides may be fractionated, such as by reverse phase chromatography or basic reverse phase chromatography. Fractions may be combined for practice of the methods of the disclosure. In some aspects, one or more fractions, including the combined fractions, are subject to phosphopeptide enrichment, including phospho-enrichment by affinity chromatography and/or binding, ion exchange chromatography, chemical derivatization, immunoprecipitation, co-precipitation, or a combination thereof. The entirety or a portion of one or more fractions, including the combined fractions and/or phospho-enriched fractions, may be subject to mass spectrometry. In some aspects, the raw mass spectrometry data may be processed and normalized using at least one relevant bioinformatics tool.

### F. Additional Assay Methods

Amplification primers or hybridization probes can be prepared to be complementary to a genomic region, biomarker, probe, or oligo described herein. The term “primer” or “probe” as used herein, is meant to encompass any nucleic acid that is capable of priming the synthesis of a nascent nucleic acid in a template-dependent process and/or pairing with a single strand of an oligo of the disclosure, or portion thereof. Typically, primers are oligonucleotides from ten to twenty and/or thirty nucleic acids in length, but longer sequences can be employed. Primers may be provided in double-stranded and/or single-stranded form, although the single-stranded form is preferred.

The use of a probe or primer of between 13 and 100 nucleotides, particularly between 17 and 100 nucleotides in length, or in some aspects up to 1-2 kilobases or more in length, allows the formation of a duplex molecule that is both stable and selective. Molecules having complementary sequences over contiguous stretches greater than 20 bases in length may be used to increase stability and/or selectivity of the hybrid molecules obtained. One may design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

In one aspect, each probe/primer comprises at least 15 nucleotides. For instance, each probe can comprise at least or at most 20, 25, 50, 75, 100, 125, 150, 175, 200, 225, 250, 275, 300, 325, 350, 400 or more nucleotides (or any range derivable therein). They may have these lengths and have a sequence that is identical or complementary to a gene described herein. Particularly, each probe/primer has relatively high sequence complexity and does not have any ambiguous residue (undetermined “n” residues). The probes/primers can hybridize to the target gene, including its RNA transcripts, under stringent or highly stringent conditions. It is contemplated that probes or primers may have inosine or other design implementations that accommodate recognition of more than one human sequence for a particular biomarker.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

A nucleic acid array can comprise at least 1, 2, 3, 4, 5, 6, 7, 8, 9, 10, 15, 20, 25, 30, 35, 40, 45, 50, 60, 70, 80, 90, 100, 150, 200, 250 or more different polynucleotide probes, which may hybridize to different and/or the same biomarkers. Multiple probes for the same gene can be used on a single nucleic acid array. Probes for other disease genes can also be included in the nucleic acid array. The probe density on the array can be in any range. In some aspects, the density may be or may be at least 50, 100, 200, 300, 400, 500 or more probes/cm2 (or any range derivable therein).

Specifically contemplated are chip-based nucleic acid technologies such as those described by Hacia et al. (1996) and Shoemaker et al. (1996). Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization (see also, Pease et al., 1994; and Fodor et al, 1991). It is contemplated that this technology may be used in conjunction with evaluating the expression level of one or more cancer biomarkers with respect to diagnostic, prognostic, and treatment methods.

Certain aspects may involve the use of arrays or data generated from an array. Data may be readily available. Moreover, an array may be prepared in order to generate data that may then be used in correlation studies.

### II. Detection Kits and Systems

One can recognize that based on the methods described herein, detection reagents, kits, and/or systems can be utilized to detect the SNP and/or the CNA related to the genetic signature for diagnosing an individual (the detection either individually or in combination). The reagents can be combined into at least one of the established formats for kits and/or systems as known in the art. As used herein, the terms “kits” and “systems” refer to aspects such as combinations of at least one SNP detection reagent, for example at least one selective oligonucleotide probe, and at least one CNA detection reagent, for example at least one PCR primer. The kits could also contain other reagents, chemicals, buffers, enzymes, packages, containers, electronic hardware components, etc. The kits/systems could also contain packaged sets of PCR primers, oligonucleotides, arrays, beads, or other detection reagents. Any number of probes could be implemented for a detection array. In some aspects, the detection reagents and/or the kits/systems are paired with chemiluminescent or fluorescent detection reagents. Particular aspects of kits/systems include the use of electronic hardware components, such as DNA chips or arrays, or microfluidic systems, for example. In specific aspects, the kit also comprises one or more therapeutic or prophylactic interventions in the event the individual is determined to be in need of.

In specific aspects, the kit may comprise one or both of a composition for detecting a polymorphism and a composition for detecting a CNA. The composition in the kit for detecting the polymorphism may be selected from the group consisting of oligonucleotide, one or more primers suitable for amplifying the polymorphism, one or more sequencing reagents, and a combination thereof. The composition in the kit for detecting the CNA may be selected from the group consisting of one or more primers suitable for amplifying the polymorphism, one or more sequencing reagents, one or more arrays, and a combination thereof.

Certain aspects of the present disclosure also concern kits containing compositions of the disclosure or compositions to implement methods disclosed herein. In some aspects, kits can be used to evaluate one or more biomarkers. In certain aspects, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein. In some aspects, there are kits for evaluating biomarker activity in a cell.

Kits may comprise components, which may be individually packaged or placed in a container, such as a tube, bottle, vial, syringe, or other suitable container means.

Individual components may also be provided in a kit in concentrated amounts; in some aspects, a component is provided individually in the same concentration as it would be in a solution with other components. Concentrations of components may be provided as 1×, 2×, 5×, 10×, or 20× or more.

Kits for using probes, synthetic nucleic acids, nonsynthetic nucleic acids, and/or inhibitors of the disclosure for prognostic or diagnostic applications are included as part of the disclosure. Specifically contemplated are any such molecules corresponding to any biomarker identified herein, which includes nucleic acid primers/primer sets and probes that are identical to or complementary to all or part of a biomarker, which may include noncoding sequences of the biomarker, as well as coding sequences of the biomarker.

In certain aspects, negative and/or positive control nucleic acids, probes, and inhibitors are included in some kit aspects. In addition, a kit may include a sample that is a negative or positive control for methylation of one or more biomarkers.

Any aspect of the disclosure involving specific biomarker by name is contemplated also to cover aspects involving biomarkers whose sequences are at least 80, 81, 82, 83, 84, 85, 86, 87, 88, 89, 90, 91, 92, 93, 94, 95, 96, 97, 98, 99% identical to the mature sequence of the specified nucleic acid.

Aspects of the disclosure include kits for analysis of a pathological sample by assessing biomarker profile for a sample comprising, in suitable container means, two or more biomarker probes, wherein the biomarker probes detect one or more of the biomarkers identified herein. The kit can further comprise reagents for labeling nucleic acids in the sample. The kit may also include labeling reagents, including at least one of amine-modified nucleotide, poly(A) polymerase, and poly(A) polymerase buffer. Labeling reagents can include an amine-reactive dye.

### III. Administration of Therapeutic Compositions

Aspects herein relate to, at least in part, administration of therapeutic interventions to a patient with cancer. In certain aspects, the therapeutic intervention comprises radiotherapy. In certain aspects, the therapeutic intervention comprises an immune checkpoint blockade (ICB) therapy.

The therapy provided herein may comprise administration of a combination of therapeutic interventions, such as an immunotherapy, for example a checkpoint inhibitor therapy, and a radiotherapy. The therapies may be administered in any suitable manner known in the art. The ICB therapy and the radiotherapy may be administered sequentially (at different times) or concurrently (at the same time or approximately the same time; also “simultaneously” or “substantially simultaneously”).

In some aspects, the ICB therapy and the radiotherapy are administered simultaneously. In some aspects, the ICB therapy and the radiotherapy are administered sequentially. In some aspects, the ICB therapy is administered before administering the radiotherapy. In some aspects, the ICB therapy is administered after administering the radiotherapy. In some aspects, a first dose of the ICB therapy is administered before administering the radiotherapy and further dose(s) of the ICB therapy are administered after administering the radiotherapy.

Aspects of the disclosure relate to compositions and methods comprising therapeutic compositions. The different therapies may be administered in one composition or in more than one composition, such as 2 compositions, 3 compositions, or 4 compositions. Various combinations of the agents may be employed.fdays

The therapeutic agents of the disclosure may be administered by the same route of administration or by different routes of administration. In some aspects, the ICB therapy is administered intratumorally, intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

In some aspects, a single dose of the immunotherapy, such as the ICB therapy, is administered. In some aspects, multiple doses of the immunotherapy are administered. In some aspects, the immunotherapy is administered at a dose of between 1 mg/kg and 5000 mg/kg. In some aspects, the immunotherapy is administered at a dose of at least, at most, or about 11,2, 3,4, 5, 6, 7, 8,9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501,502,503,504,505,506,507,508,509,510,511,512,513,514,515,516,517,518,519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, or 5000 mg/kg, or any range derivable therein. In certain aspects, an immunotherapy is not administered to the patient.

In some aspects, the radiotherapy administered to the subject provides irradiation in a dose range of 0.5 Gy to 60 Gy. In some aspects, the radiotherapy administered to the subject provides irradiation at a dose of at least, at most, or about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 Gy. In some aspects, the radiotherapy is administered in a single dose. In some aspects, the radiotherapy is administered in a fractionated dose over a period of time of not more than one week. In some aspects, the radiotherapy is delivered in a fractionated dose over a period of time of not more than three days. In certain aspects, the radiotherapy is not administered to the patient.

The quantity to be administered, both according to number of treatments and unit dose, depends on the treatment effect desired. The term “therapeutic benefit” or “therapeutically effective” as used throughout this application refers to anything that promotes or enhances the well-being of the subject with respect to the medical treatment of cancer. This includes, but is not limited to, a reduction in the frequency or severity of the signs or symptoms of a disease. For example, treatment of cancer may include but is not limited to total or partial remission of the cancer. Treatment of cancer may also refer to prolonging survival of a subject with a cancer. The term “therapeutically effective amount” refers to an amount sufficient to produce a desired therapeutic result.

In the practice in certain aspects, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain aspects, the effective dose of the pharmaceutical composition is one which can provide a blood level of about 1 μM to 150 μM. In another aspect, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other aspects, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 M or any range derivable therein. In certain aspects, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

### A. Cancer Therapy

In some aspects, the method further comprises administering a cancer therapy to the patient. The cancer therapy may be chosen based on the expression level measurements, alone or in combination with the clinical risk score calculated for the patient. In some aspects, the cancer therapy comprises a local cancer therapy. In some aspects, the cancer therapy excludes a systemic cancer therapy. In some aspects, the cancer therapy excludes a local therapy. In some aspects, the cancer therapy comprises a local cancer therapy without the administration of a system cancer therapy. In some aspects, the cancer therapy comprises an immunotherapy, which may be an immune checkpoint therapy. Any of these cancer therapies may also be excluded. Combinations of these therapies may also be administered.

The term “cancer,” as used herein, may be used to describe a solid tumor, metastatic cancer, or non-metastatic cancer. In certain aspects, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus. In some aspects, the cancer is recurrent cancer. In some aspects, the cancer is Stage I cancer. In some aspects, the cancer is Stage II cancer. In some aspects, the cancer is Stage III cancer. In some aspects, the cancer is Stage IV cancer.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

### IV. Checkpoint Inhibitors and Radiotherapy

In some aspects, the therapy comprises immune checkpoint inhibitors (which may be referred to herein as immune checkpoint blockade therapies). In certain aspects, the therapy comprises radiotherapy. Certain aspects are further described below.

### A. PD-1, PD-L1, and PD-L2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PD-L1 on epithelial cells and tumor cells. PD-L2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PD-L1 activity.

Alternative names for “PD-1” include CD279 and SLEB2. Alternative names for “PD-L1” include B7-H1, B7-4, CD274, and B7-H. Alternative names for “PD-L2” include B7-DC, Btdc, and CD273. In some aspects, PD-1, PD-L1, and PD-L2 are human PD-1, PD-L1 and PD-L2.

In some aspects, the PD-1 inhibitor is a molecule that inhibits the binding of PD-1 to its ligand binding partners. In a specific aspect, the PD-1 ligand binding partners are PD-L1 and/or PD-L2. In another aspect, a PD-L1 inhibitor is a molecule that inhibits the binding of PD-L1 to its binding partners. In a specific aspect, PD-L1 binding partners are PD-1 and/or B7-1. In another aspect, the PD-L2 inhibitor is a molecule that inhibits the binding of PDL2 to its binding partners. In a specific aspect, a PD-L2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some aspects, the PD-1 inhibitor is an anti-PD-1 antibody (e.g., a human antibody, a humanized antibody, or a chimeric antibody). In some aspects, the anti-PD-1 antibody is selected from the group consisting of nivolumab, pembrolizumab, and pidilizumab. In some aspects, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In some aspects, the PD-L1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDIO680, also known as AMP-514, and REGN2810.

In some aspects, the immune checkpoint inhibitor is a PD-L1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PD-L2 inhibitor such as rHIgM12B7.

In some aspects, the inhibitor comprises the heavy and light chain CDRs or VRs of nivolumab, pembrolizumab, or pidilizumab. Accordingly, in one aspect, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another aspect, the antibody competes for binding with and/or binds to the same epitope on PD-1, PD-L1, or PD-L2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

### B. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some aspects, the inhibitor blocks the CTLA-4 and B7-1 interaction. In some aspects, the inhibitor blocks the CTLA-4 and B7-2 interaction.

In some aspects, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO0 1/14424).

In some aspects, the inhibitor comprises the heavy and light chain CDRs or VRs of tremelimumab or ipilimumab. Accordingly, in one aspect, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another aspect, the antibody competes for binding with and/or binds to the same epitope on PD-1, B7-1, or B7-2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

### C. Radiotherapy

In some aspects, the additional therapy or prior therapy comprises radiation, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

In some aspects, the amount of ionizing radiation is greater than 20 Gy and is administered in one dose. In some aspects, the amount of ionizing radiation is 18 Gy and is administered in three doses. In some aspects, the amount of ionizing radiation is at least, at most, or exactly 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, or 40 Gy (or any derivable range therein). In some aspects, the ionizing radiation is administered in at least, at most, or exactly 1, 2, 3, 4, 5, 6, 7, 8, 9, or 10 doses (or any derivable range therein). When more than one dose is administered, the doses may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some aspects, the amount of IR may be presented as a total dose of IR, which is then administered in fractionated doses. For example, in some aspects, the total dose is 50 Gy administered in 10 fractionated doses of 5 Gy each. In some aspects, the total dose is 50-90 Gy, administered in 20-60 fractionated doses of 2-3 Gy each. In some aspects, the total dose of IR is at least, at most, or about 20, 21, 22, 23, 24, 25, 26, 27, 28, 29, 30, 31, 32, 33, 34, 35, 36, 37, 38, 39, 40,41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 (or any derivable range therein). In some aspects, the total dose is administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein. In some aspects, at least, at most, or exactly 2, 3, 4, 5,6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40,41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some aspects, at least, at most, or exactly 1, 2, 3, 4, 5, 6, 7, 8, 9, 10, 11, or 12 (or any derivable range therein) fractionated doses are administered per day. In some aspects, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

### EXAMPLES

The following examples are included to demonstrate preferred aspects of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific aspects which are disclosed and still obtain a like or similar result without departing from the spirit and scope of the invention.

### Example 1: Tumor Aneuploidy Predicts Survival Following Immunotherapy Across Multiple Cancers

Aneuploidy, defined as an unbalanced number of chromosomes or chromosome arms, is a nearly universal feature of human cancer⁸. Recent studies have brought to light the negative impact of tumor aneuploidy on anti-tumor immunity, potentially through immune evasion⁸ via mechanisms including downregulation of PD-L1 expression⁹ and suppression of the intratumoral CD8+ T cell response¹⁰. Moreover, previous work has established elevated tumor aneuploidy as a marker of poor overall survival¹¹ and suggested aneuploidy as a biomarker of clinical outcomes¹². In this context, we recently found that aneuploidy is a strong predictor of survival in patients with non-small cell lung cancer treated with immunotherapy¹³.

Here, by reanalyzing an immunogenomic cohort comprised of 1,660 patients treated with ICIs from Samstein et al.¹, we demonstrate that tumor aneuploidy score defined as the fraction of chromosome arms afflicted by arm-level copy-number alterations-provides independent prognostic information in patients with lower TMB. We propose the use of an aneuploidy score⁸ as a complementary measure of prognosis following ICI treatment.

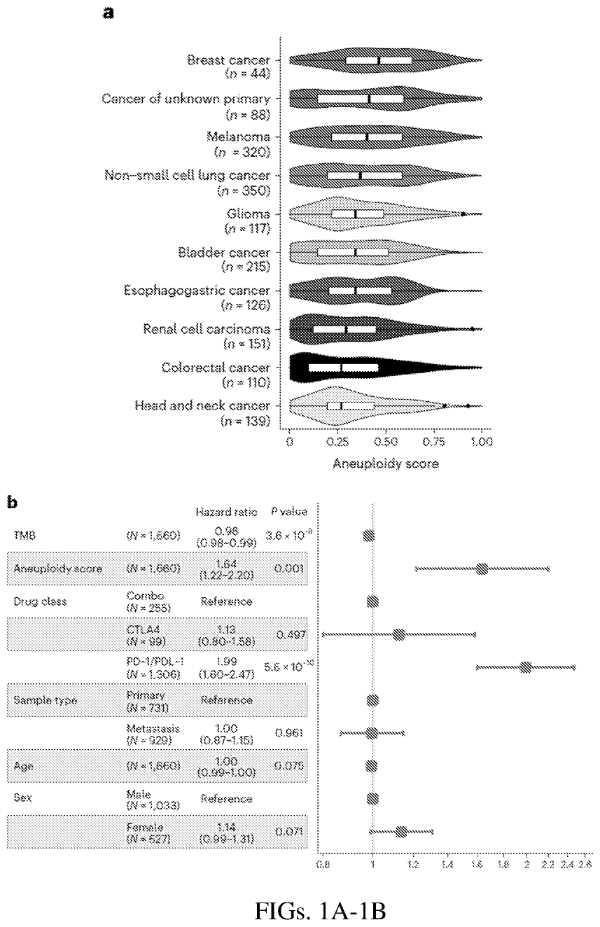

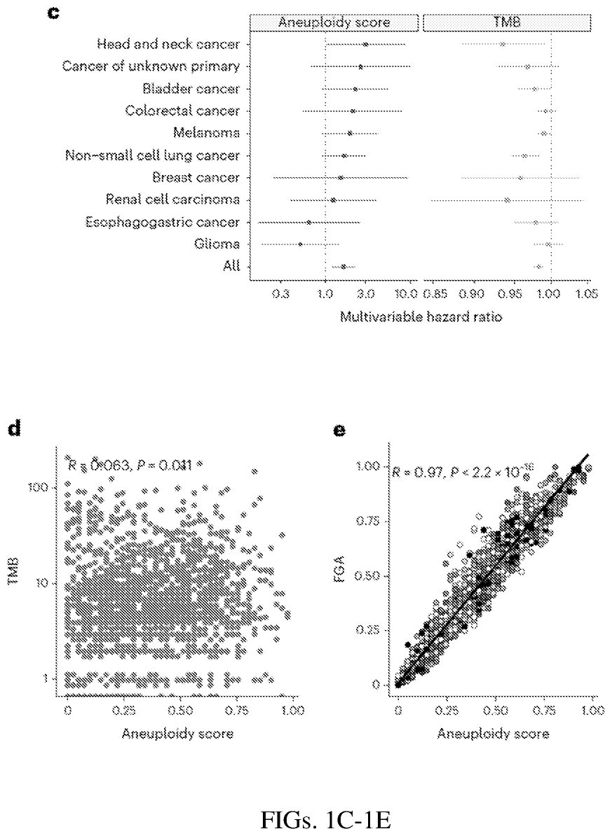

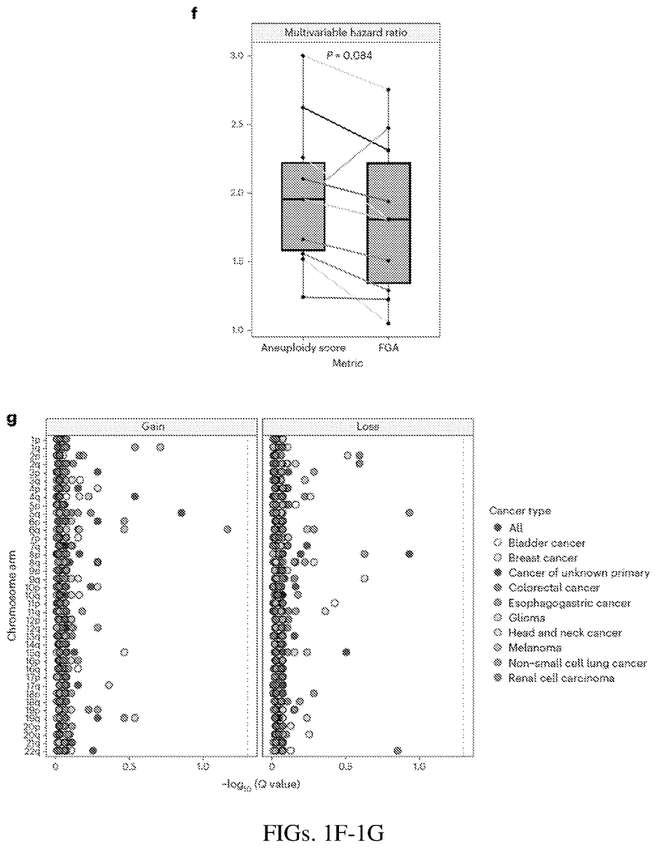

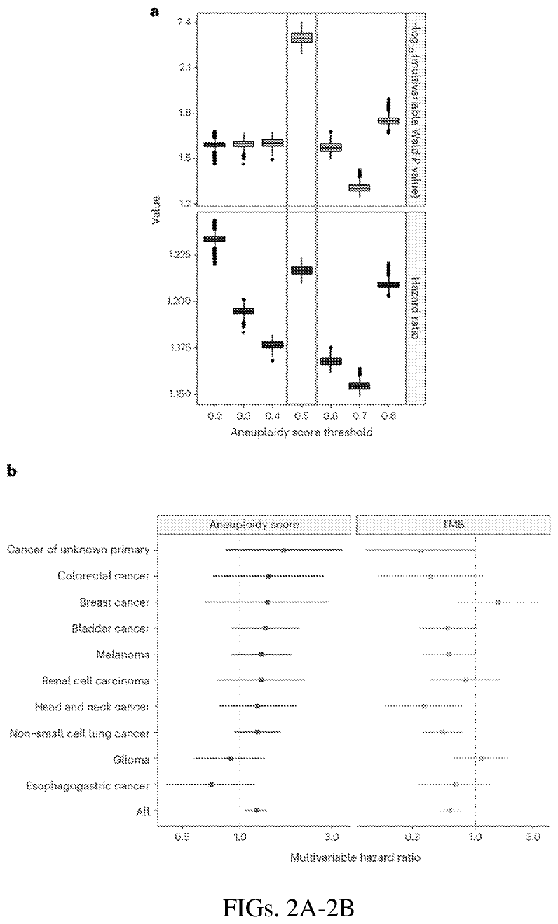

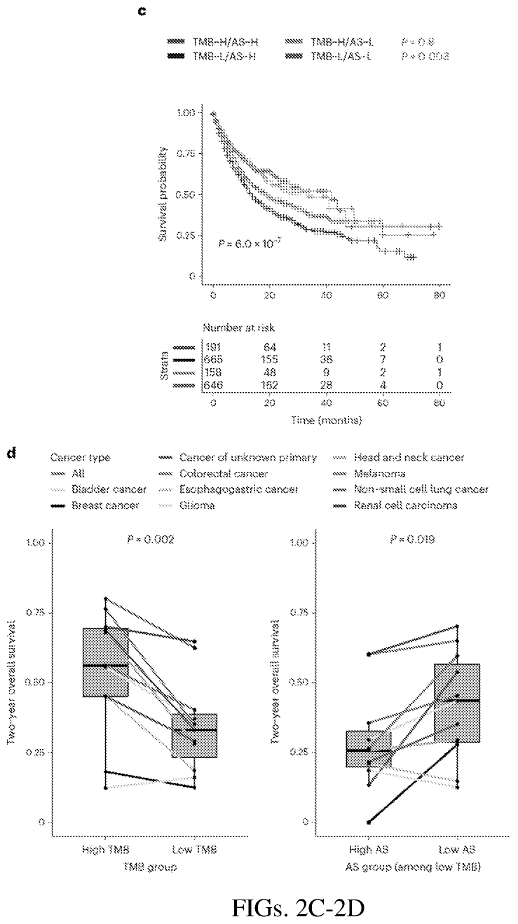

Although aneuploidy is almost uniformly present across tumor histologies (96% of samples), the distribution of aneuploidy scores varied greatly by cancer type (FIG. 1A). Across the combined cohort of 1,660 patients, a higher aneuploidy score was associated with unfavorable prognosis (hazard ratio 1.44; 95% confidence interval 1.07-1.94, P=0.014). Importantly, in a multivariable analysis with TMB and clinicopathologic features, aneuploidy score remained independently associated with overall survival across cancer types (FIGS. 1, 1C). Minimal correlation was observed between TMB and aneuploidy score, highlighting the independent prognostic value of these genomic features (Spearman rho=0.063, P=0.011, FIG. 1D). In addition, previous studies have shown that the fraction of genome encompassed by copy-number alterations (FGA) is a strong predictor of tumor immunity. FGA encompasses arm-level chromosomal events as does aneuploidy score, but also includes focal copy-number events. Therefore, aneuploidy score is a measure of true aneuploidy (an unbalanced number of chromosome arms), whereas FGA represents an overall burden of copy-number alterations. As expected, aneuploidy score was highly correlated with FGA (FIG. 1E); however, when FGA was substituted for aneuploidy score in the multivariable analysis in FIG. 1C, we found that the magnitude of hazard ratios were, on average, greater for aneuploidy score than for FGA (FIG. 1F). We also examined whether the prognostic value of aneuploidy score was predominantly impacted by specific chromosomal changes, such as loss of heterozygosity of the 9p21 locus, which harbors the gene CD274 encoding PD-L1. However, we found that no specific arm-level changes were associated with survival at a false discovery rate<0.05 when controlling for overall aneuploidy score (FIG. 1G). Taken together, these analyses supported the utility of aneuploidy score as a prognostic biomarker in the setting of immunotherapy. Because the application of continuous variables in clinical decision-making can be challenging, we determined the aneuploidy score threshold that optimally synergized with TMB to risk-stratify patients following ICI treatment by performing an analysis analogous to the approach used for TMB thresholding in Samstein et al. We tested every tenth quantile within each tumor type from the 20th to 80th percentile in a multivariable model with high TMB defined as the highest 20th percentile and ICI drug class. Using leave-one-out cross validation, we identified the 50th percentile of aneuploidy score within each tumor type as the optimal threshold of all candidate thresholds with the lowest overall P value and the only candidate with Bonferroni-corrected P<0.05. (FIGS. 2A, 2B). Notably, the same optimal aneuploidy score threshold was obtained when using the FDA-approved ten or more mutations per megabase TMB threshold in the multivariable model (P=0.003). In a Kaplan-Meier analysis dividing patients by TMB binned at the top 20th percentile and aneuploidy score binned at 50th percentile, we found that among high TMB tumors, aneuploidy score had no prognostic value (P=0.8); however, among low TMB tumors, patients with high aneuploidy score tumors experienced significantly worse outcomes following ICI treatment (P=0.003) (FIG. 2C). When stratified by tumor type, the prognostic impact of aneuploidy score within low TMB tumors was comparable with that of TMB overall in patients with colorectal cancer, bladder cancer, non-small cell lung cancer and cancer of unknown primary (FIG. 2D). By contrast, aneuploidy score exhibited a larger impact on survival for patients with low TMB breast cancer and renal cell carcinoma when compared with stratifying patients as high or low TMB alone. Notably, the largest differences in 2-year overall survival for patients with high versus low aneuploidy score among the low TMB population were observed in cancer of unknown primary (13% versus 54%), colorectal cancer (26% versus 60%) and breast cancer (0% versus 28%).

Taken together, we demonstrated that an elevated aneuploidy score is an independent and complementary predictor of overall survival for patients with low TMB tumors treated with ICIs. We established that the 50th percentile represents a threshold for defining high aneuploidy score with independently prognostic value in tumors defined as low TMB by either the lowest 80th percentile or the FDA-approved threshold of fewer than ten mutations per megabase. However, there are several important limitations. Only ten cancer types are represented in this data set; of those included, the sample sizes of several histologies, such as breast cancer and cancer of unknown primary, are relatively limited. In addition, this data set includes patients from a single institution, which may impact the generalizability of the study. Therefore, future studies, particularly prospective clinical trials, are needed to validate tumor aneuploidy as a prognostic biomarker. In addition, it will be crucial to identify synergistic treatment modalities that may help to overcome the immunoresistance in highly aneuploid tumors. Nevertheless, the study provides an important first step in understanding the role of tumor aneuploidy in mediating response to immunotherapy among tumors with low TMB. Importantly, like TMB, aneuploidy scores can be calculated from routine tumor-only targeted DNA sequencing, as recently published¹⁴, indicating that this biomarker can be implemented alongside TMB using existing clinical sequencing infrastructure. These findings demonstrate a potential role for tumor aneuploidy to guide the personalization of immunotherapeutic approaches for patients with diverse cancers.

### Example 2: Methods for Certain Aspects

Data for the Samstein et al. cohort were downloaded from cBioPortal^{1,15}. Segmented copy-number data were downloaded from AACR Project GENIE v.7.1. Arm-level somatic copy-number alterations were called using ASCETS¹⁴ v.1.1 on the normalized copy-number segmentation files using a threshold for calling amplifications and deletions of ±0.1. The aneuploidy score⁸ for a sample was defined as the fraction of evaluable arms (ASCETS call of AMP, DEL, NEUTRAL or NC) afflicted by arm-level somatic copy-number alterations (AMP or DEL). FGA was defined as the fraction of genomic territory covered by copy-number segments with |log₂ copy ratio|>0.1.

Overall survival was defined as the time from initiation of immunotherapy to death. Patients were censored at the time of last follow-up. Survival analysis was performed using the survminer v.0.4.9 and survival v.3.4.0 packages, utilizing the survdif( ) function in R to compute a log-rank P value for Kaplan-Meier survival analyses. For univariable and multivariable Cox proportional hazards analysis, the coxph( ) function was used. Unadjusted or adjusted hazard ratios for survival were reported as appropriate.

Leave-one-out cross validation was performed to determine an optimal threshold for defining high versus low aneuploidy. Candidate thresholds of 0.2 to 0.8 in steps of 0.1 were analyzed. For each threshold, a Cox proportional hazards survival model of binarized aneuploidy score with TMB (high defined as highest 20th percentile) and drug class was constructed. This process was repeated n times, where n is the cohort size (1,660), leaving out one unique patient in each iteration. For comparisons of two continuous variables, a Spearman correlation was used. Unless otherwise specified, all tests were performed in R [0123]v.4.1.1 and were two-tailed. P<0.05 was considered significant. Correction for multiple testing was performed using the Bonferroni method via qvalue v.2.28.0 package in R. The top and bottom edges of a boxplot represent the first and third quartiles, respectively; the center line represents the median; whiskers extend to the farthest data points that do not represent outliers (within 1.5× the interquartile range); outliers are plotted as points above and below the box-and-whisker plot; n=1,660 biologically independent samples. Squares at midpoints of forest plots represent point estimates of hazard ratios; bars represent 95% confidence intervals. No statistical method was used to predetermine sample size. One ‘skin cancer-nonmelanoma’ sample was excluded from the analyses because it was the only sample of this histology. The experiments were not randomized. The investigators were not blinded to allocation during experiments and outcome assessment.

### Example 3: Highly Aneuploid Non-Small Cell Lung Cancer Shows Enhanced Responsiveness To Concurrent Radiation And Immune Checkpoint Blockade

Lung cancer remains the leading cause of cancer-related deaths in the United States. Immune checkpoint blockade (ICB) has revolutionized the management of metastatic non-small cell lung cancer (NSCLC) lacking targetable driver mutations; however, only a fraction of patients respond to treatment¹. Mounting preclinical evidence indicates that radiotherapy can exert immunomodulatory effects to augment immunotherapeutic responses²⁻⁶. There is also evidence to suggest that anti-programmed cell death protein 1 (anti-PD-1) and anti-cytotoxic T-lymphocyte-associated protein 4 (anti-CTLA-4) immune checkpoint inhibitors enhance both local and distant tumor responses to radiotherapy⁷. The potential synergy between radiotherapy and ICB has led to over 500 clinical trials in various malignancies, based on the hypothesis that there is a synergistic antitumor interaction between radiotherapy and ICB. Although there is evidence that consolidation ICB after radiotherapy can improve outcomes in some settings^{8,9}, this has also been demonstrated after surgery and therefore does not prove the existence of a synergistic interaction of ICB and radiotherapy. Furthermore, limited biological evidence exists in patients to optimize the potential interaction between local radiotherapy and the immune system with regard to radiation dose, fractionation, timing, and target organs.

At present, a critical clinical question is whether and how radiotherapy and ICB can be combined in a manner that will improve outcomes for patients. Moreover, identification of biomarkers of response will be instrumental in selecting patients who are most likely to benefit from combination therapy. Currently, programmed death-ligand 1 (PD-L1) expression, tumor mutational burden (TMB), effector T cell gene signature expression and the number of tumor neoantigens have been demonstrated to predict anti-PD-1 and/or anti-CTLA-4 response¹⁰⁻¹⁴. However, it remains unknown whether any of these or other biomarkers predict responses to the combination of radiotherapy and ICB.

We conducted a randomized clinical trial designed to evaluate the safety and efficacy of combination ICB plus multisite ablative radiotherapy as a first-line treatment for patients with biomarker-unselected metastatic NSCLC. As a secondary analysis of the trial, we present a comprehensive molecular characterization of matched pretreatment and on-treatment tumor biopsies in this unique dataset to delineate the effect of radiotherapy and ICB timing on the tumor immunogenomic milieu. We define treatment-specific changes in the response to ablative radiotherapy as opposed to ablative radiotherapy with concurrent ICB. Importantly, we nominate a genomic biomarker that predicts clinical benefit and improved survival following concurrent radiotherapy and ICB in metastatic NSCLC. These findings serve as a framework to guide the personalization of combinatorial radiotherapy and ICB strategies in patients with metastatic NSCLC and may be applicable to other human cancers.

### Results

### Cohort Characteristics

A total of 37 patients with metastatic NSCLC were randomized to receive concurrent or sequential stereotactic body radiotherapy (SBRT) and ipilimumab plus nivolumab (ipi/nivo) immunotherapy as part of the randomized phase I trial to evaluate concurrent or sequential ipilimumab, nivolumab and stereotactic body radiotherapy in patients with stage IV non-small cell lung cancer (the COSINR study, NCT03223155)¹⁵. The CheckMate 227 trial previously demonstrated that ipi/nivo improved clinical outcomes compared with nivo (±platinum-doublet chemotherapy) or chemotherapy alone^{13,16}. With the future goal of determining whether SBRT further improves response rates to and survival following ipi/nivo, the COSINR phase I study evaluated the safety of combining multisite metastasis-directed SBRT to ipi/nivo in the first-line treatment of metastatic NSCLC. As recently reported, concurrent treatment resulted in fewer toxicities than sequential treatment¹⁵. In addition, concurrent treatment demonstrated a favorable objective response rate (ORR) and overall survival (OS) compared with the ipi/nivo arm of the CheckMate 227 trial (ORR, 44% versus 36%; 1-year OS, 84% versus 62%; 2-year OS, 62% versus 40%). The recently completed phase II component of the trial evaluated the efficacy of concurrent therapy in an expanded clinical cohort.

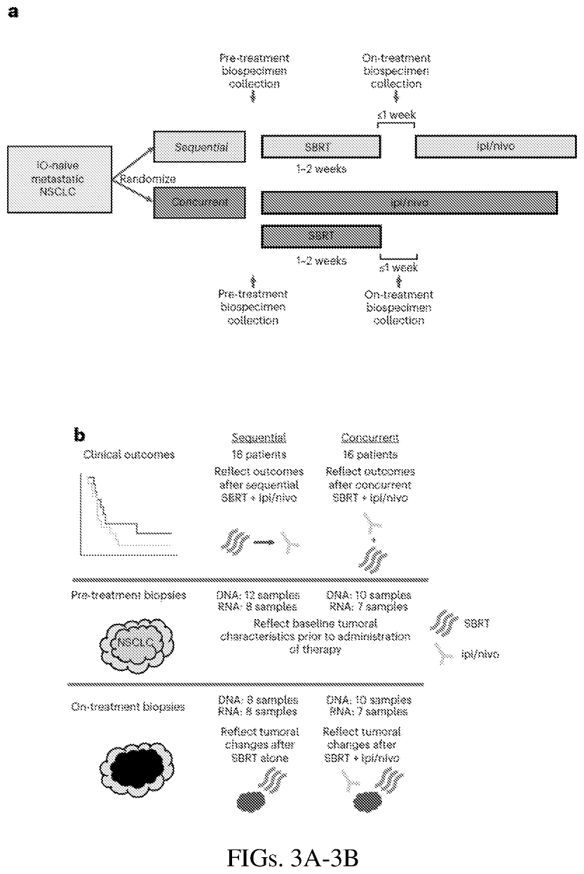

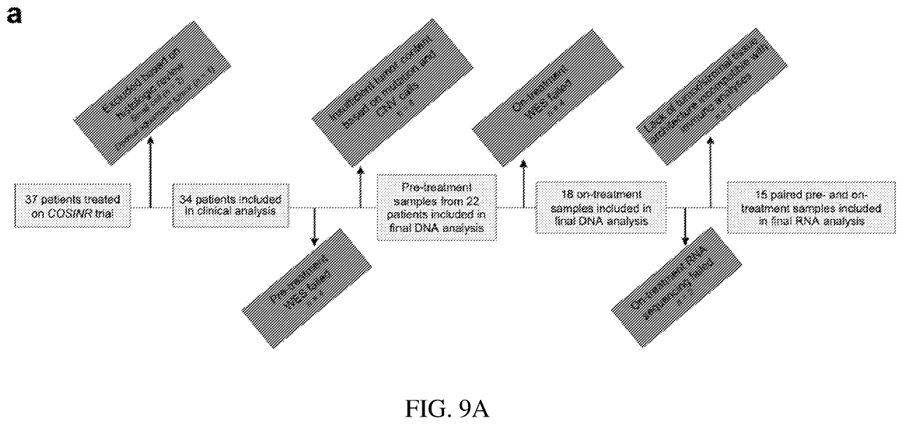

For all patients, pretreatment tumor biopsies were obtained prior to the administration of any therapy. Within the sequential arm, an on-treatment tumor biopsy was obtained after completion of SBRT, but prior to administration of ipi/nivo. Within the concurrent arm, an on-treatment biopsy was obtained after completion of SBRT and one cycle of ipi/nivo (FIG. 3A). The obtained pretreatment and on-treatment tumor biopsies were of the same irradiated metastatic lesion. Therefore, by comparing changes in matched tumor biopsies during treatment, we investigated the effect of SBRT versus SBRT+ipi/nivo on the tumor microenvironment (FIG. 3B). After review of pathologic and genomic data, 22 patients successfully underwent whole exome sequencing of matched tumor (pretreatment with or without on-treatment) and normal samples; total RNA sequencing (RNA-seq) of matched pretreatment and on-treatment tumor samples was successful for a subset of 15 patients (FIG. 9A). A schematic of the analytical framework is shown in FIG. 9B.

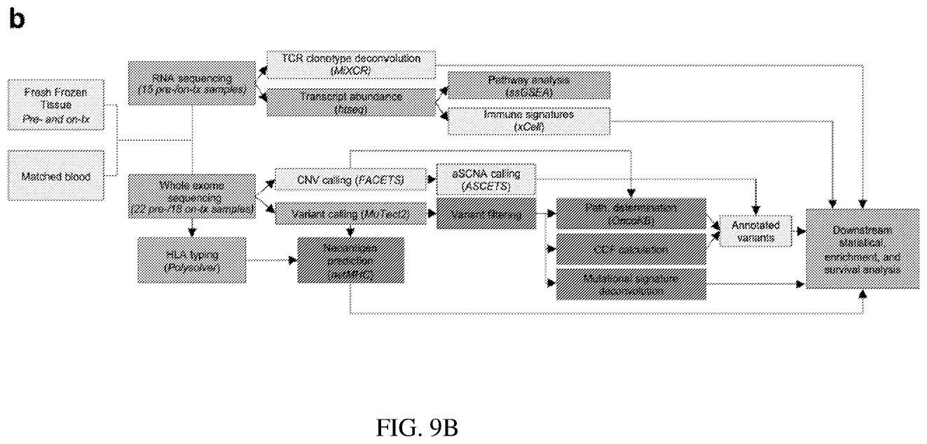

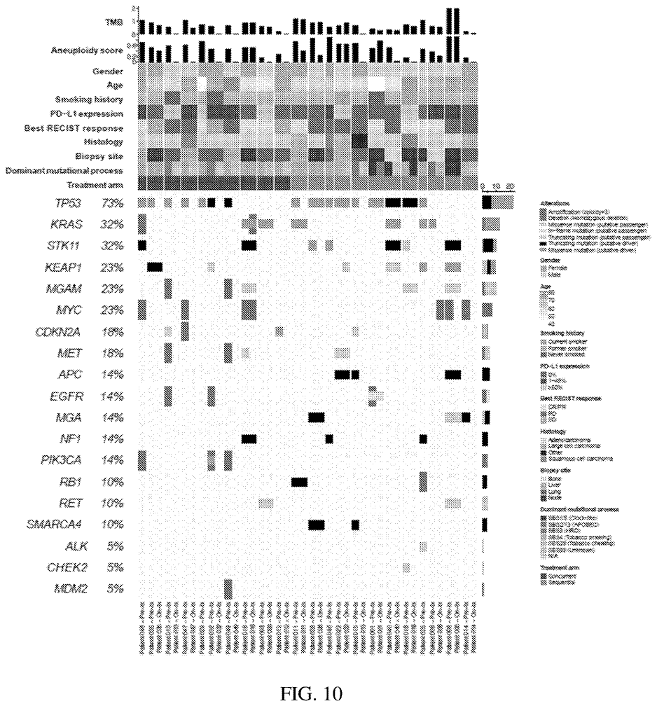

Overall clinical and genomic characteristics of the cohort are presented in FIG. 10. Clinicopathological characteristics were balanced between the concurrent and sequential treatment arms (Supplementary Table 17). A positive smoking history was associated with improved progression-free survival (PFS), whereas a greater number of disease sites and the presence of liver metastasis were associated with worse PFS; a larger number of disease sites was also associated with worse OS (FIG. 11A). At a median follow-up of 17 months, there were no differences in PFS or OS between the treatment arms in the overall cohort (FIG. 11B) or the subset of 22 patients in the molecular analysis (FIG. 11C). In this context, we investigated whether a distinct molecular subset of patients experienced differential outcomes following treatment.

### Treatment-Unique Immunogenomic Changes

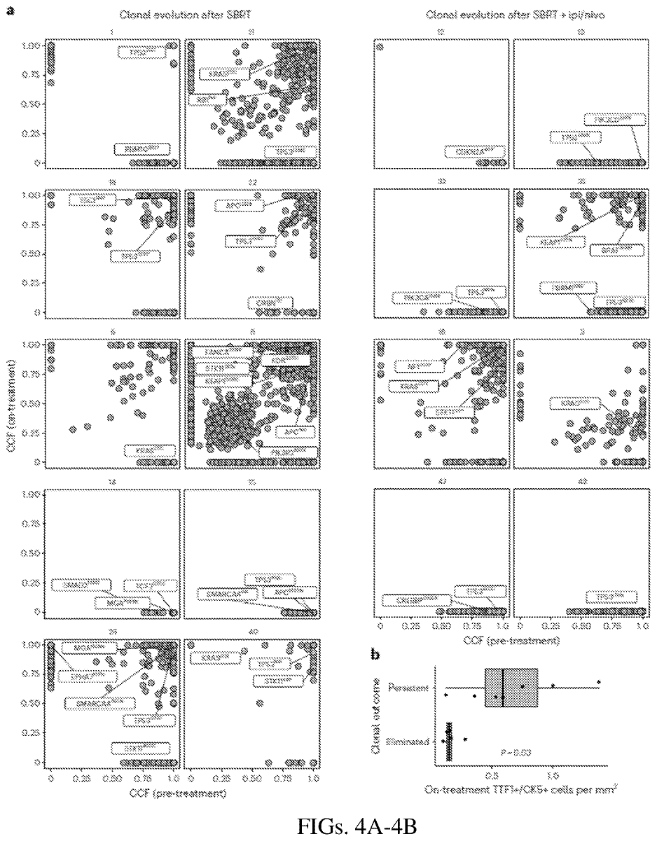

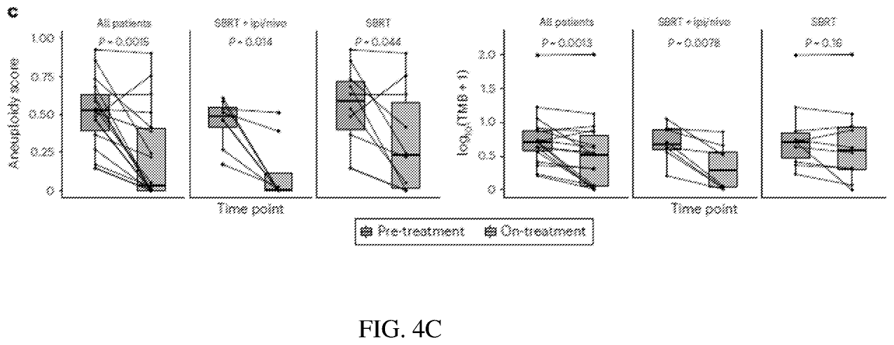

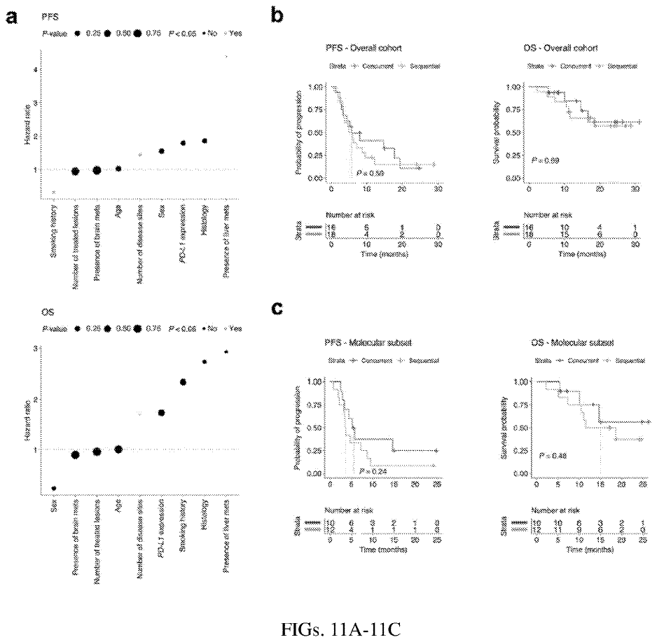

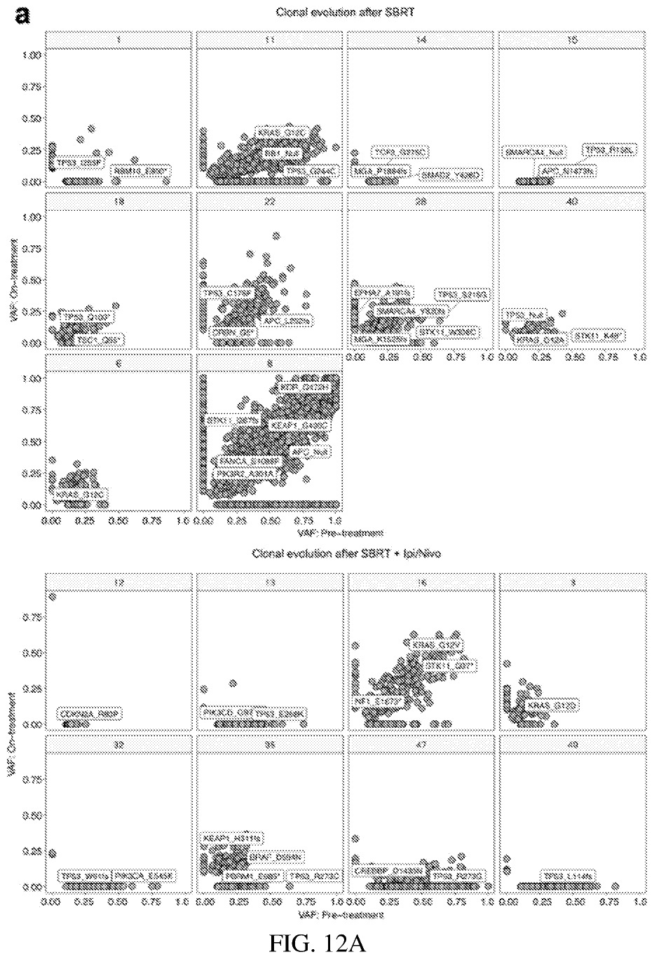

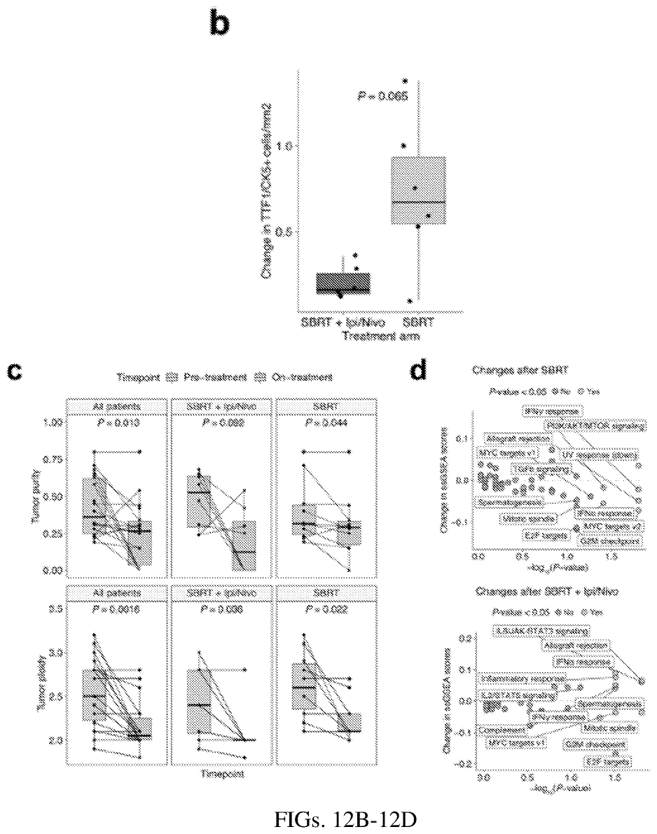

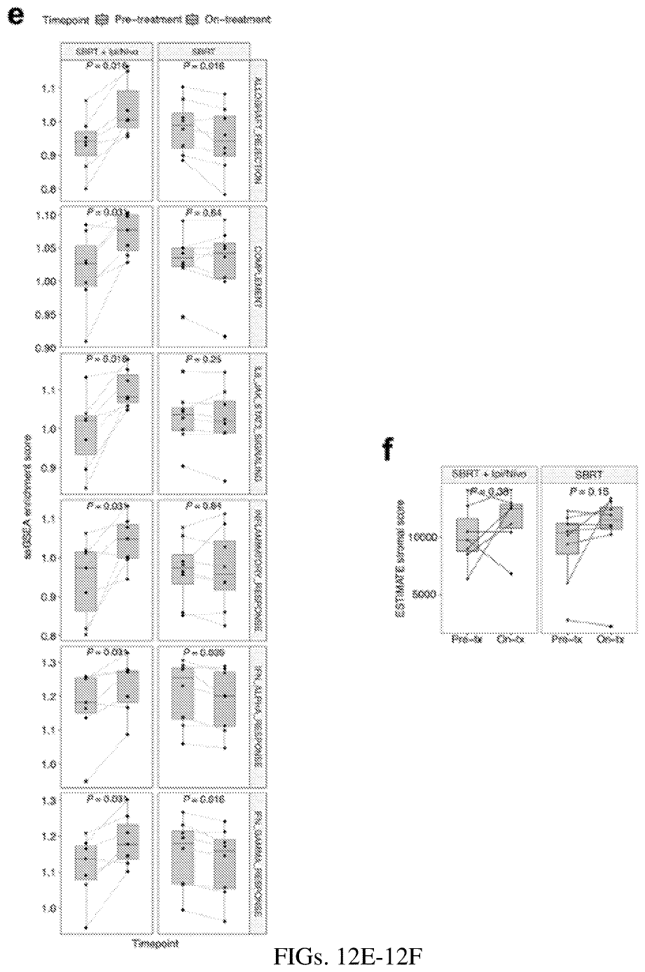

We first characterized the differential genomic and transcriptomic effects of SBRT versus SBRT+ipi/nivo, which have not been previously reported in metastatic NSCLC. At the genomic level, we found substantial clonal evolution of somatic mutations after both SBRT and SBRT+ipi/nivo (FIG. 4A and FIG. 12A). The majority of tumors treated with SBRT+ipi/nivo (5 out of 8) showed complete loss of tumor variants during therapy, suggesting complete or near-complete pathologic responses. The remaining tumors (3 out of 8) retained a clone with a core group of variants accompanied by at least one driver mutation alongside loss of existing and acquisition of new, somatic mutations. We investigated whether these genomic findings correlated with histologic reduction in the density of TTF1+/CK5+ cancer cells by performing multiplexed immunofluorescence analysis of matched formalin-fixed paraffin-embedded (FFPE) tumor specimens, which were independently obtained to validate the immunogenomic findings from fresh frozen tumor analysis. Tumors in which all genomic variants were eliminated on therapy had a significantly lower density of TTF1+/CK5+ cells than tumors that retained genomic variants on treatment (Wilcoxon P=0.03) (FIG. 4B), whereas no differences were observed between groups at baseline (P=0.27). By contrast, after SBRT, most tumors (8 out of 10) exhibited the latter phenotype of clonal persistence (FIG. 4A), suggesting less effective tumor cell elimination with SBRT compared with SBRT+ipi/nivo, as corroborated by multiplexed immunofluorescence analysis (FIG. 12B). In addition, TMB and aneuploidy score, which is defined as the fraction of evaluable chromosomal arms that were amplified or deleted, were correlated at baseline (Spearman's rho=0.54, P=0.0095) and decreased on treatment across both treatment arms (TMB median, 4.4 versus 1.2 mutations per megabase, paired Wilcoxon P=0.0013; aneuploidy score median, 0.53 versus 0.04, paired Wilcoxon P=0.0015); however, these results were primarily driven by changes following SBRT+ipi/nivo rather than SBRT alone (FIG. 4C). The reduction in TMB and aneuploidy score was also accompanied by a tumoral diploidization and reduction in purity (FIG. 12C), suggesting that the loss of genomic variants during treatment was due to tumor cell elimination and subsequent enrichment of immune cells as opposed to a reduction of genomic instability within individual cancer cells. Similarly, clonal persistence in this setting reflected ineffective local treatment response.

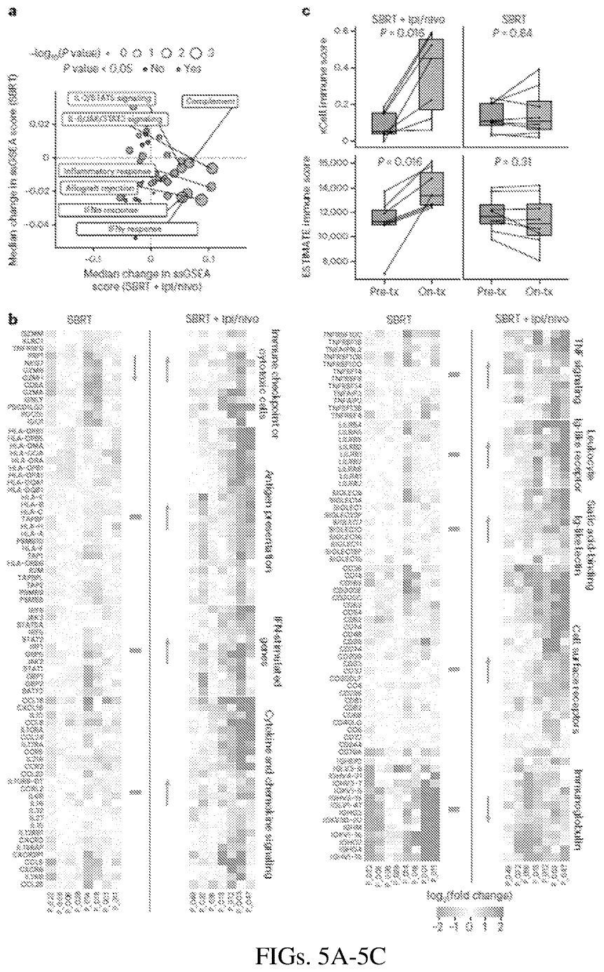

Changes in gene expression were also dramatically different after SBRT compared with SBRT+ipi/nivo. SBRT+ipi/nivo upregulated, whereas SBRT decreased, immune signaling through IFNα, IFNγ, IL-6/JAK/STAT3 and inflammatory pathways (FIGS. 5A, 12D, 12E). In addition, SBRT+ipi/nivo, but not SBRT, decreased expression of G2/M cell cycle checkpoint, mitotic spindle, and E2F-dependent and MYC-dependent proliferation pathways (FIG. 12D). The inventors investigated whether these changes were related to an increase in stromal cells; however, neither SBRT nor SBRT+ipi/nivo increased the ESTIMATE (estimation of stromal and immune cells in malignant tumor tissues using expression data) stromal score (FIG. 12F). In addition, there was no correlation between the change in single-sample gene set enrichment analysis (ssGSEA) hallmark pathway scores and the change in ESTIMATE stromal scores during treatment, which suggested that treatment-induced changes in cell cycle and proliferation pathways following SBRT+ipi/nivo were due to reprogramming of remaining cancer cells rather than dilution with stromal cells.

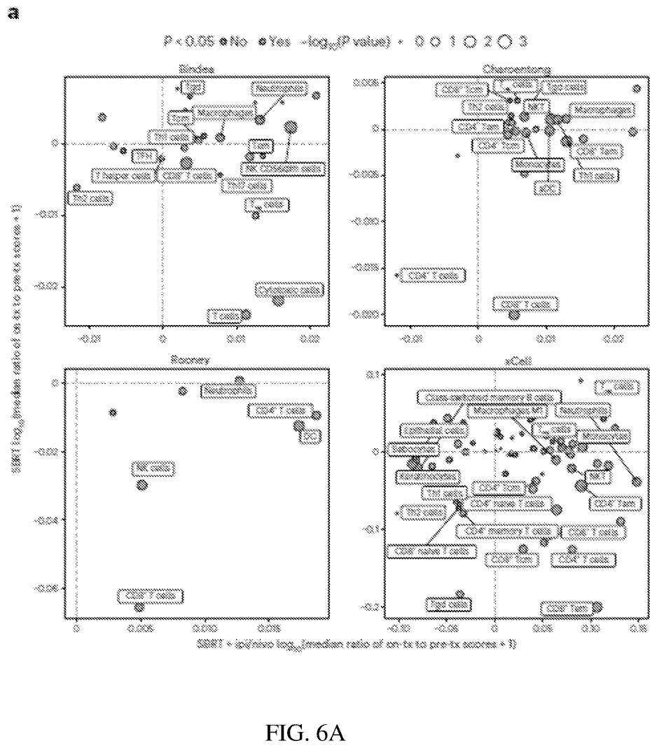

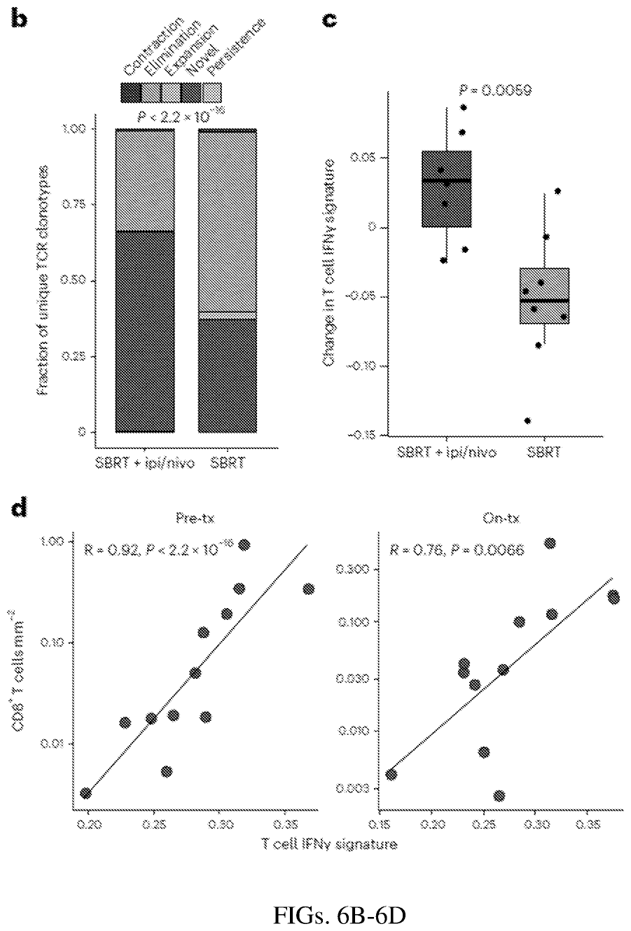

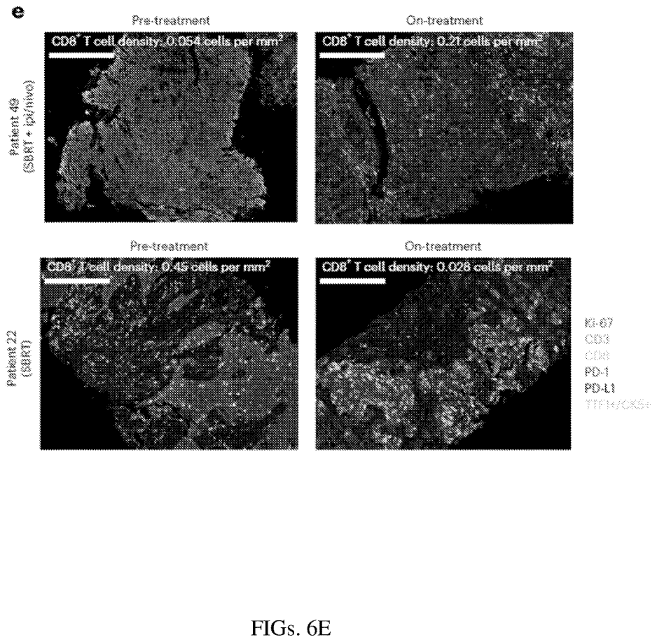

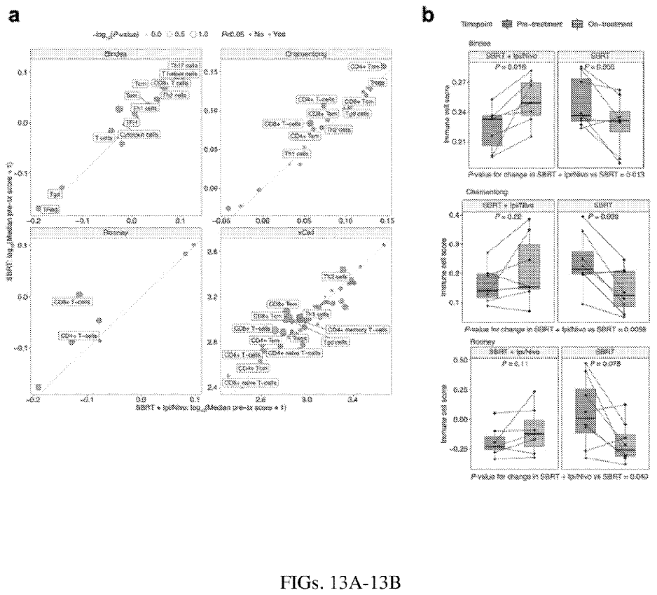

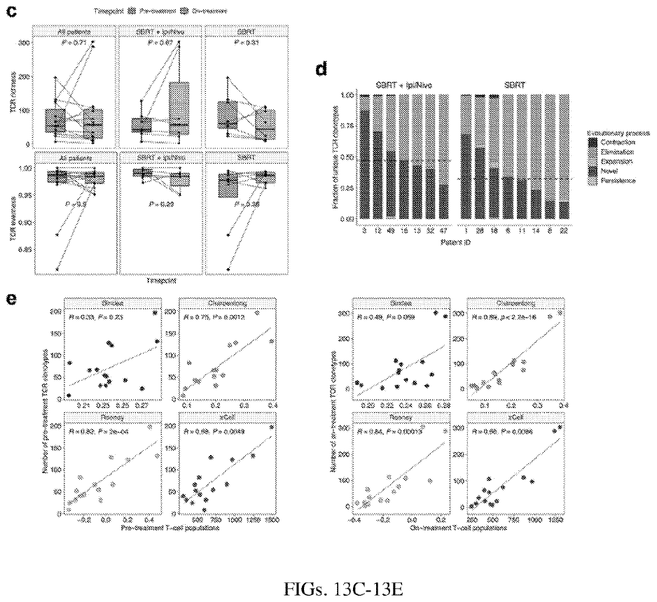

In concert with these findings, there was an upregulation of immune genes involved in antigen presentation, interferon response, cytokine and chemokine signaling, and effector T cell function by SBRT+ipi/nivo (FIG. 5B). By contrast, SBRT suppressed expression of cytotoxic T cell genes. In addition, SBRT+ipi/nivo, but not SBRT, increased immune content as indicated by upregulation of xCell and ESTIMATE immune scores (FIG. 5C). Moreover, although analysis of immune cell signatures using xCell¹⁷ showed no baseline differences between treatment arms (FIG. 13A), following treatment the inventors observed a reduction in T cell expression signatures, particularly those representing CD8⁺ populations, after SBRT, whereas SBRT+ipi/nivo increased signatures of these key immune populations (FIGS. 6A, 13B).

The differential effect on effector T cells was further supported by intratumoral T cell receptor (TCR) analysis using MiXCR¹⁸. Overall, there were no changes in TCR richness or evenness in either treatment arm (FIG. 13C); however, despite the lack of numerical changes in TCR diversity, individual TCRs showed a high degree of evolution. After SBRT, 641 out of 678 (94.5%) of TCRs present at baseline were eliminated on therapy, and 401 novel clonotypes were detected in the tumor. After SBRT+ipi/nivo, a similar portion of TCRs (380 out of 398, 95.5%) were eliminated on therapy; however, twofold more (764) novel clonotypes were present on therapy, suggesting that ICB facilitated the recruitment of a larger number of novel T cells into the tumor microenvironment (FIGS. 6B, 13D). Furthermore, TCR diversity correlated with the abundance of CD8⁺ T cells as represented by the xCell immune cell signatures (FIG. 13E). Owing to the single sampling of TCRs in tumor samples and limited availability of fresh frozen tissue, the inventors were unable to account for potential heterogeneity of TCR clonotypes across different tumoral regions^{19,20} or perform dedicated TCR sequencing. In addition, the inventors further analyzed the cytolytic activity of the intratumoral T cells using an eight-gene effector T cell IFNγ-associated signature previously validated to predict ICB response in NSCLC¹⁴ and found a downregulation of the effector T cell signature after SBRT but an upregulation after SBRT+ipi/nivo (FIG. 6C). Importantly, although the T cell IFNγ-associated signature negatively correlated with FACETS tumor purity (Spearman's rho=−0.52, P=0.008), when changes in FACETS tumor purity were controlled for in a multivariable logistic regression model, the inventors found that treatment arm was an independent predictor of change in the IFNγ-associated signature (P=0.000594). This result indicated that the T cell IFNγ-associated signature was specifically upregulated following SBRT+ipi/nivo in the setting of overall changes in tumor purity.

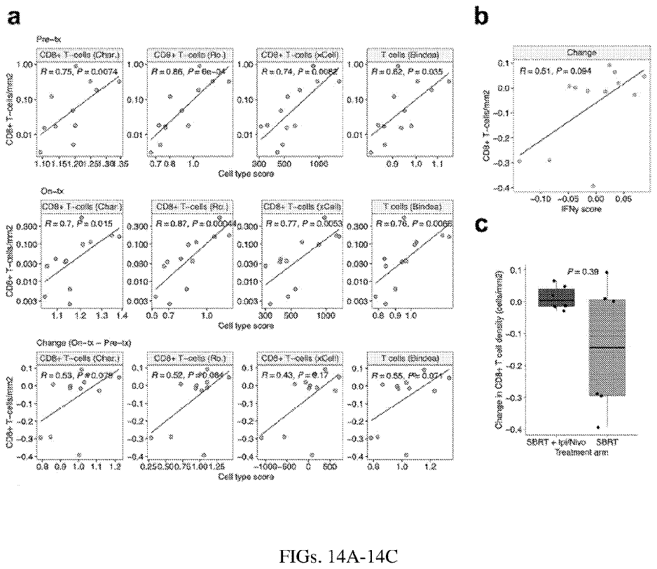

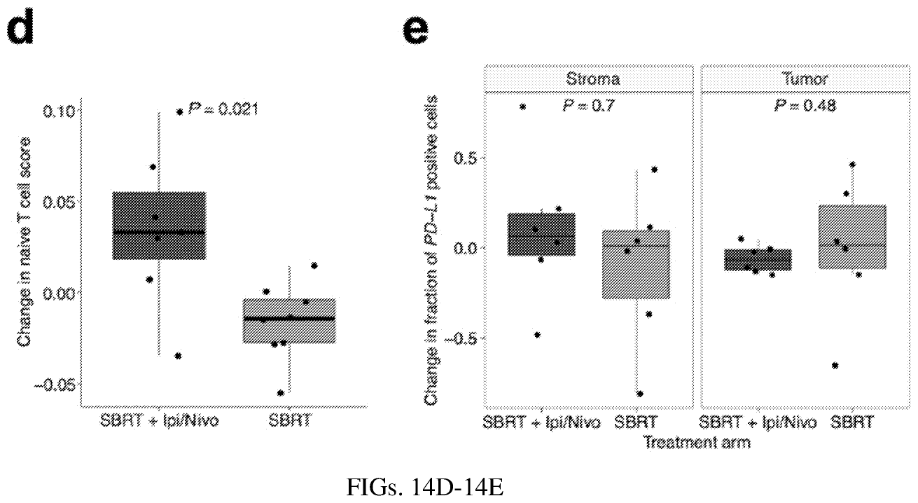

The density of CD8⁺ T cells strongly correlated with both the expression of CD8⁺ T cell signatures (FIG. 14A) and the expression of the T cell IFNγ-associated signature (FIG. 6D). This finding corroborated the result indicating that the transcriptomic signatures were reliable measures of activated, tumor-infiltrating CD8⁺ T cells. In the response to treatment, the change in CD8⁺ T cell density moderately correlated with changes in the expression of CD8⁺ T cell and T cell IFNγ-associated signatures (FIGS. 14A, 14B). This was likely due to the fact that most tumors treated with SBRT+ipi/nivo showed an increase in or stable CD8⁺ T cell density, whereas most tumors treated with SBRT demonstrated a reduction in CD8⁺ T cell density (FIGS. 6E, 14C). Notably, intratumoral T cells showed a naive phenotype following SBRT+ipi/nivo as opposed to SBRT (FIG. 14D). Collectively, these findings demonstrated that SBRT+ipi/nivo more effectively eliminated tumor cells and amplified adaptive immunity compared with SBRT alone. In addition, these findings suggested that the stability in CD8⁺ T cell density following SBRT+ipi/nivo reflected a balance in the treatment-induced infiltration of activated CD8⁺ T cells and the loss of pre-existing tumor-resident CD8⁺ T cells.

### Established Immunotherapy Biomarkers

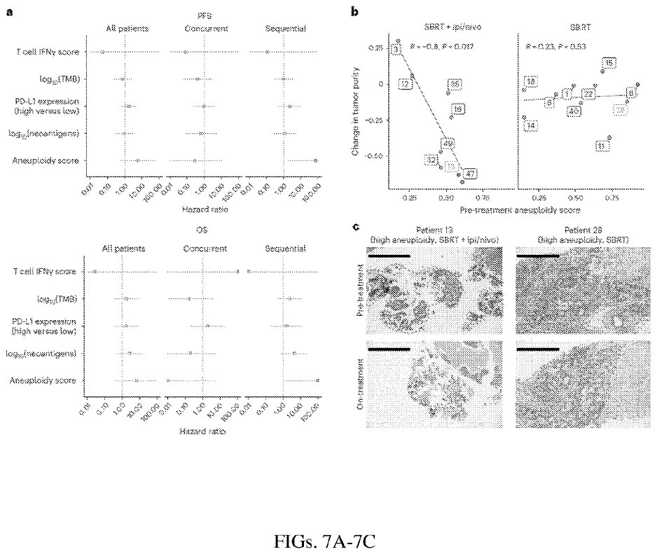

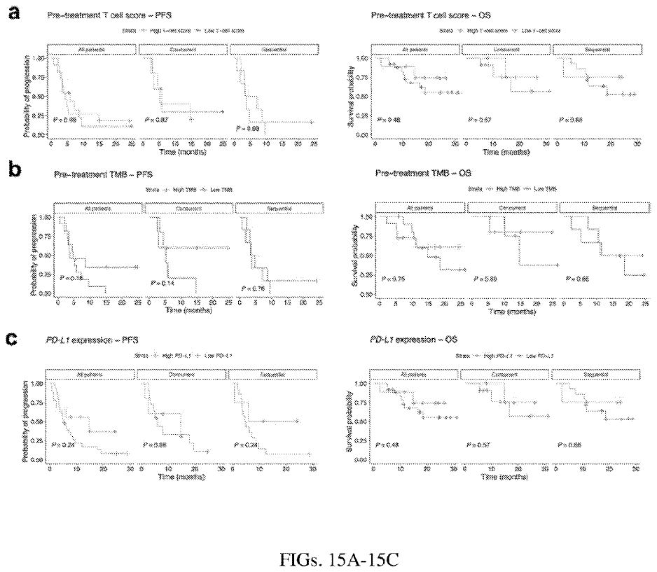

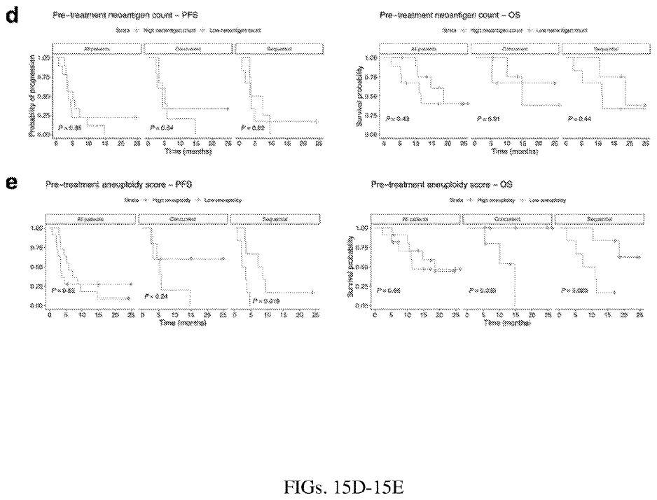

Several biomarkers, including T cell IFNγ signature expression¹⁴, TMB, PD-L1 expression and neoantigen load, have been previously established as predictors of ICB response. Therefore, the inventors examined whether these biomarkers were predictive of outcome in the context of SBRT+ipi/nivo. The inventors found that none of these biomarkers were associated with PFS or OS, in the case of the entire cohort and in either treatment arm (FIGS. 7A, 15A-15D). No specific mutations, gene-level copy number alterations (CNAs) or arm-level somatic CNAs (aSCNAs) were associated with PFS or OS, albeit the study may have been underpowered to detect such associations. In addition, the inventors observed no differences in the percentage of PD-L1-positive tumor or stromal cells at baseline, or in the response to treatment, between treatment arms (FIG. 14E). Neither pretreatment nor change in the percentage of PD-L1-positive tumor or stromal cells was associated with survival across all patients or in either treatment arm. Therefore, the inventors examined whether additional biomarkers could serve as predictors of survival following SBRT+ipi/nivo.

Recent studies suggest that highly aneuploid tumors show inferior responses to ICB, potentially due to mechanisms of immune evasion, such as downregulation of PD-L1 expression²¹ and reduction of tumor-infiltrating CD8⁺ T cells²². In the cohort, pretreatment aneuploidy score was differentially predictive of adverse outcomes in patients receiving sequential, but not concurrent, SBRT+ipi/nivo (OS, hazard ratio 94.9, 95% confidence interval 1.80-7633.4, P=0.04; PFS, hazard ratio 74.4, 95% confidence interval 1.93-2922, P=0.021) (FIGS. 7A, 15E). Aneuploidy score remained independently predictive of poor PFS (P=0.018) and OS (P=0.08) in patients receiving sequential therapy after controlling for TMB, as well as baseline prognostic clinical and pathological features (FIG. 11A) (multivariable PFS (controlling for smoking history, number of disease sites and presence of liver metastases), P=0.04; multivariable OS (controlling for number of disease sites), P=0.06). These findings identified tumor aneuploidy as a potential biomarker of SBRT+ipi/nivo response in metastatic NSCLC.

The inventors examined the relationship between baseline tumor aneuploidy and the change in tumor content following treatment, based on the previous findings that aneuploidy score and tumor purity decreased on-treatment. The inventors found no correlation between baseline aneuploidy score and change in tumor purity after SBRT, although most samples demonstrated a small decrease in tumor purity following SBRT (Spearman's rho=0.23, P=0.53) (FIG. 7B), probably secondary to tumor cell killing. By contrast, baseline aneuploidy score was highly negatively correlated with the change in tumor purity after SBRT+ipi/nivo (Spearman's rho=−0.80, P=0.017) (FIG. 7B), suggesting that highly aneuploid tumors were more effectively eliminated with SBRT+ipi/nivo than with SBRT. In support of these findings, pathologic review of tumor samples from highly aneuploid tumors demonstrated near-complete tumor responses following SBRT+ipi/nivo, but not following SBRT (FIG. 7C).

### Aneuploidy Predicts Response to Radiotherapy Plus ICB in an Independent Cohort

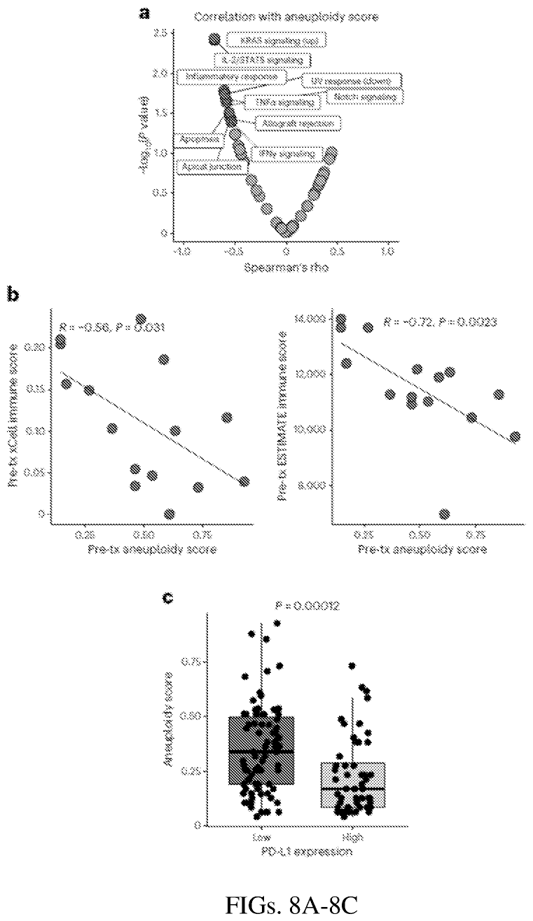

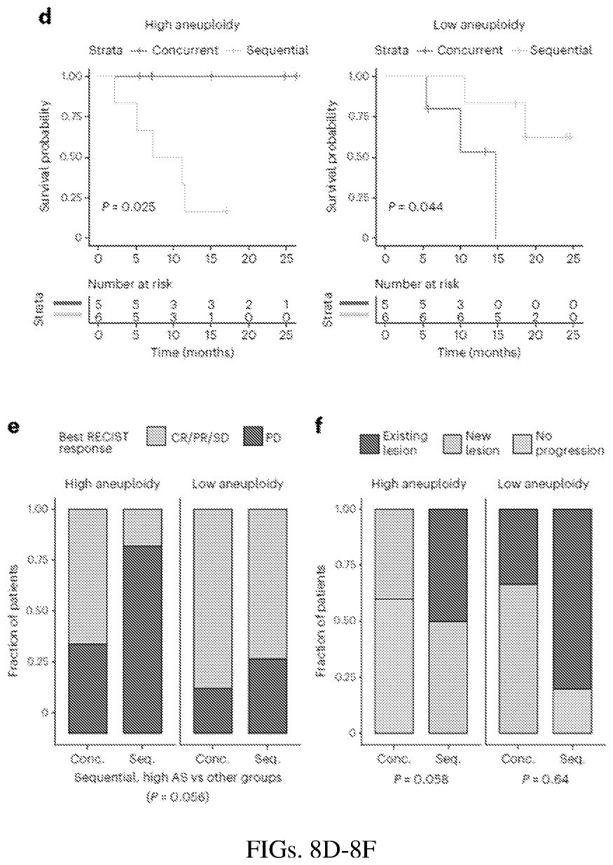

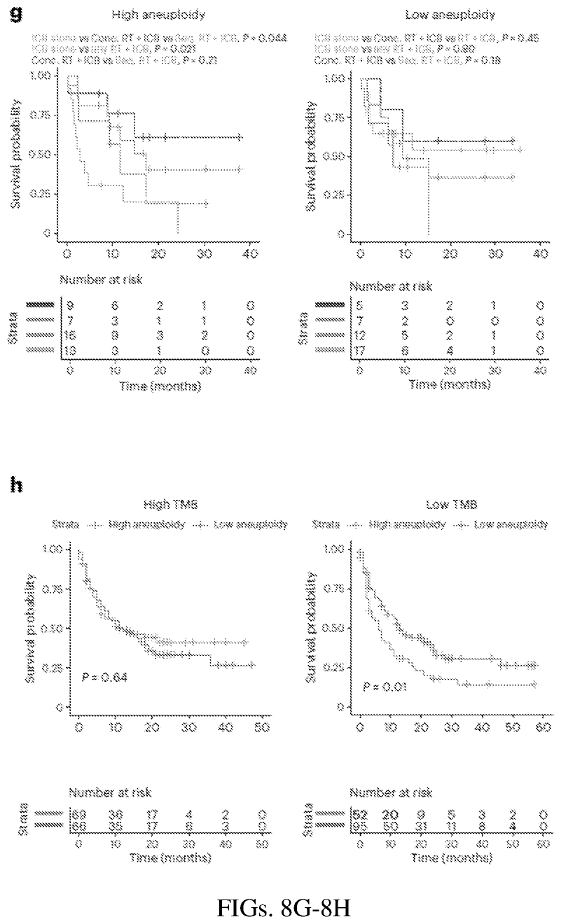

Our finding that baseline aneuploidy was predictive of local tumor responses and survival led us to hypothesize that a pretreatment aneuploidy score could serve as a biomarker of the response to radiotherapy+ICB and survival. Aneuploidy score showed a moderate correlation with the FACETS fraction of genome altered (Spearman's rho=0.51, P=0.000724), as aneuploidy score only encompasses arm-level changes, whereas the fraction of genome altered also includes focal events. Pretreatment aneuploidy score was negatively associated with immune signaling programs, including IFNγ, TNFα, IL-2/STAT5 and inflammatory pathways (FIG. 8A) and ESTIMATE immune and xCell immune scores (FIG. 8B). In addition, patients with low PD-L1 expression, which is associated with reduced response to ICB, showed higher aneuploidy scores (FIG. 8C). Previous studies have demonstrated that aneuploidy is associated with TP53 mutation, TMB and expression of proliferation genes²³. Notably, TP53 mutation, TMB and expression of hallmark proliferation signatures (including mitotic spindle, G2/M checkpoint, E2F targets and MYC targets) were not associated with clinical outcomes in the cohort. In addition, aneuploidy score was not associated with other clinicopathologic features at baseline. However, the lack of association between specific genomic alterations or proliferation signatures and survival was likely to have been limited by the modest sample size of the study; therefore, the inventors cannot conclude that aneuploidy score is independently associated with clinical outcomes.

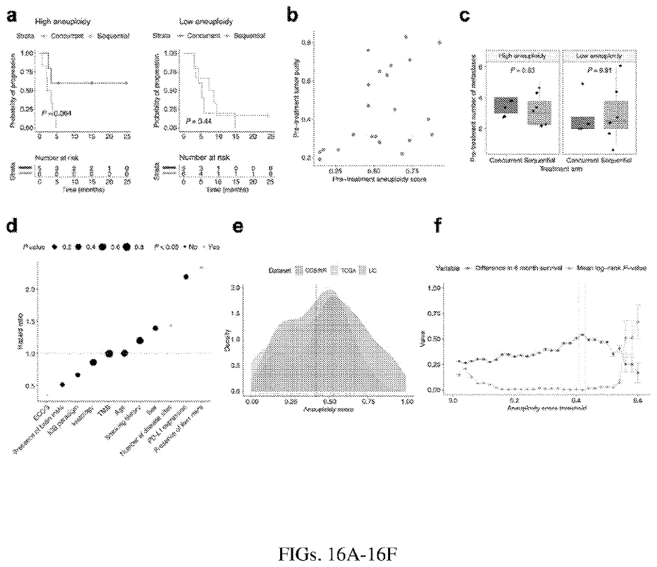

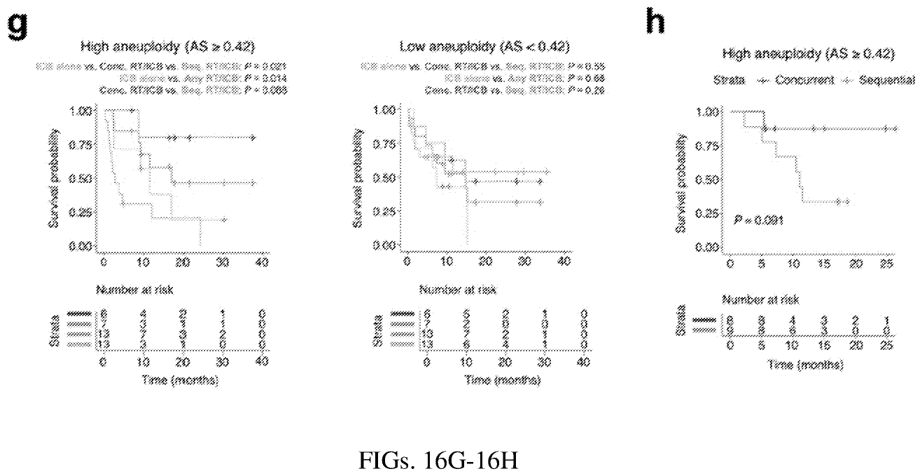

The inventors further examined the relationship between aneuploidy score and survival. The inventors found that patients whose tumors exhibited high aneuploidy (aneuploidy score≥cohort median, 0.54; interquartile range, 0.32-0.78) had improved survival when treated with concurrent as opposed to sequential SBRT+ipi/nivo (12-month OS, 100% versus 17%, log-rank P=0.025) (FIG. 8D). PFS followed similar trends to OS (FIG. 16A). By contrast, this relationship was not observed in patients with less aneuploid tumors (12-month PFS, 20% versus 17% (concurrent versus sequential), log-rank P=0.44; 12-month OS, 53% versus 83% (concurrent versus sequential), log-rankP=0.044) (FIG. 8D). Although there were three deaths in the concurrent arm and two deaths in the sequential arm for low-aneuploidy tumors, one death in the concurrent arm was that of a patient for whom there was no evidence of active cancer. Instead, the death was attributed to baseline pulmonary disease. All other deaths of patients included in this study were preceded by disease progression. When the inventors examined cancer-specific survival for low-aneuploidy tumors, there was no difference between the two treatment arms (log-rank P=0.24).

The inventors further examined the treatment-related effects on nonirradiated tumor sites. Within the subset of high-aneuploidy tumors, distant tumor responses in nonirradiated lesions occurred in 17% of patients treated with sequential therapy and 60% of patients treated with concurrent therapy, which was similar to the percentages seen in low aneuploidy tumors irrespective of treatment arm (FIG. 8E). In addition, high-aneuploidy tumors treated with concurrent therapy were less likely to experience disease progression in an existing unirradiated site compared with tumors treated with sequential therapy (FIG. 8F). Importantly, the associations between high aneuploidy score and decreased survival were not confounded by baseline tumor purity or the number of disease sites (FIGS. 16B, 16C). These findings suggested that in highly aneuploid tumors, concurrent therapy increased abscopal responses in nonirradiated lesions compared with sequential therapy, and improved survival. The inventors examined whether the effect of tumor aneuploidy on the differential responses to radiotherapy and ICB could be validated in independent clinical metastatic NSCLC cohorts. The inventors reasoned that concurrent radiotherapy and ICB benefits patients whose tumors demonstrate high aneuploidy due to their adverse baseline prognosis. To test this hypothesis, the inventors investigated an independent contemporary clinical cohort of 58 patients with metastatic NSCLC who underwent next-generation targeted genomic sequencing prior to ICB initiation at the institution (The University of Chicago (UC) cohort)²⁴ (Methods). Patients received either ICB alone (anti-PD-1 or anti-PD-L1 inhibitor±cytotoxic chemotherapy) or ICB with radiotherapy to at least one extracranial disease site during (concurrent) or preceding or following (sequential) ICB. The compositions of clinical and pathological factors were balanced across the COSINR and UC cohorts (Supplementary Table 19). Consistent with the hypothesis, the inventors found that after ICB alone, high aneuploidy score (aneuploidy score≥cohort median, 0.40; interquartile range, 0.21-0.53) was associated with reduced tumor responses (complete response, partial response or stable disease, 10% versus 50% (high versus low aneuploidy score), Fisher's exact P=0.07) and OS compared with low aneuploidy score (12-month OS, 33% versus 56% (high aneuploidy score versus low aneuploidy score), log-rank P=0.06). Importantly, patients with highly aneuploid tumors who received radiotherapy+ICB had higher 12-month survival than those who received ICB alone (59% versus 31%, log-rank P=0.021) (FIG. 8G). Moreover, among the patients who received radiotherapy+ICB, those who received the two modalities concurrently experienced better outcomes than those who received them sequentially, although this did not reach statistical significance (12-month OS, 76% versus 38%, P=0.21). By contrast, no survival improvement was observed in adding radiotherapy to ICB in patients with low aneuploidy score tumors (FIG. 8G). Among known prognostic factors, worse Eastern Cooperative Oncology Group (ECOG) performance status (2 or 3 versus 0 or 1), a greater number of disease sites and the presence of liver metastases were associated with adverse OS in the UC cohort (FIG. 16D). However, in a multivariable model that includes these variables, the addition of radiotherapy to ICB (versus ICB alone) remained independently predictive of improved OS in patients with highly aneuploid tumors (aneuploidy score≥cohort median, Wald P=0.016).

To identify an optimal threshold to define high aneuploidy, the inventors performed a leave-one-out cross-validation analysis comparing data from patients who received ICB alone with that of patients who received radiotherapy+ICB in the UC cohort. The inventors defined the optimal aneuploidy score threshold in the UC cohort and not in the COSINR dataset because the distribution of aneuploidy scores was more right skewed in the COSINR cohort than in the UC cohort and in a third cohort of 500 lung adenocarcinomas from The Cancer Genome Atlas (TCGA)²⁵ (UC versus TCGA Kolmogorov-Smirnov P=0.26; UC or TCGA versus COSINR P=0.025) (FIG. 16E). Therefore, using the UC cohort allowed us to maximize generalizability of an optimal threshold to other datasets. The inventors determined that an aneuploidy score threshold of 0.42 represented the optimal cutoff in the UC cohort (6-month OS, 85% versus 31% (radiotherapy+ICB versus ICB only), log-rank P=0.014) (FIG. 16F, 16G). Importantly, applying this cross-validated aneuploidy score threshold to the COSINR cohort also demonstrated promise as a threshold for high aneuploidy score (12-month OS, 88% versus 33% (concurrent arm versus sequential arm), log-rank P=0.09) (FIG. 16H).

Last, the inventors examined the relationship between aneuploidy score, TMB and survival in an independent cohort of 350 patients with metastatic NSCLC who received ICB at the Memorial Sloan Kettering Cancer Center (MSKCC)¹⁰. In a multivariable Cox proportional hazards model, TMB and aneuploidy score independently predicted OS (TMB, hazard ratio 0.96, Wald P=0.00016; aneuploidy score, hazard ratio 2.25, P=0.014 (per unit increase)). The inventors proposed that a high aneuploidy score could independently identify a subset of patients with metastatic NSCLC with an adverse prognosis and split patients by the median TMB to compare the OS of patients whose tumors exhibited a high aneuploidy score (≥0.42) with those whose tumors exhibited a low aneuploidy score (<0.42). The data demonstrated that among low TMB tumors, high aneuploidy score tumors showed significantly worse OS (12-month OS, 30% versus 52% (high aneuploidy score versus low aneuploidy score), log-rank P=0.01), further supporting the utility of the high aneuploidy score threshold as a biomarker of ICB response in metastatic NSCLC (FIG. 8H). Taken together, the findings suggest that high aneuploidy score metastatic NSCLCs show a deleterious baseline prognosis with ICB alone and derive a large clinical benefit from the concurrent use of radiotherapy and ICB.

### Discussion

Our findings challenge the prevailing view that ablative radiotherapy induces a favorable intratumoral adaptive immune response, reveal differential immunogenomic consequences of ablative radiotherapy versus concomitant radiotherapy and ICB, and establish high tumor aneuploidy as a potential biomarker of radiotherapy and ICB response. By comprehensively analyzing the molecular features of matched pretreatment and on-treatment tumor biopsies from patients treated on a randomized clinical trial, the inventors determined that SBRT combined with ipi/nivo increased the expression of adaptive immune and cytotoxic T cell gene programs, increased TCR clonotypic diversity and improved tumor cell elimination over SBRT alone. These findings support preclinical studies that demonstrated upregulation of major histocompatibility complex class I expression, augmentation of immunogenic cell death and cytokine expression, and improvements in CD8⁺ effector T cell function with combination radiotherapy and ICB^{2-7,26}. However, the findings demonstrated that SBRT alone was insufficient in metastatic NSCLC to induce local immune augmentation that has been suggested in previous studies^{25,27}. The lack of induced innate and adaptive immunity by SBRT in the study may be due to the timing of the on-treatment biopsy. Most preclinical studies have measured cytokine induction and CD8⁺ T cell priming at relatively short intervals following radiotherapy (for example, 10-14 days), and the apparent lack of a positive immune response observed here (median 27.5 days between biopsies) may be related to the durability of immunity induced by radiotherapy alone rather than lack of immune activation by radiotherapy.

The inventors identified elevated tumor aneuploidy score as a predictor of survival following radiotherapy and ICB in metastatic NSCLC, which supports recent findings in patients with metastatic melanoma treated with ICB²² and NSCLC treated with radiotherapy²⁸. Extending these findings, the inventors demonstrated that radiotherapy to extracranial disease sites concomitant with, but not before or after, ICB improves the adverse baseline prognosis of patients with highly aneuploid tumors, which the inventors suggest is due to augmentation of local and distant tumor immunity. By contrast, no survival benefit was detected with the addition of radiotherapy to ICB in patients with less aneuploid tumors. The inventors propose that tumors exhibiting elevated aneuploidy derive the greatest benefit from the addition of concurrent radiotherapy and ICB because concurrent therapy elicits a more rapid and deeper local tumor response (that is, greater clonal elimination combined with increased immune infiltration) compared with sequential therapy, which ultimately affects systemic disease response and survival. On a practical level, aneuploidy can be readily obtained from targeted genomic sequencing panel data using existing methods²⁹, as demonstrated in the UC validation cohort. Future trials of radiotherapy and ICB are needed to validate aneuploidy as a biomarker.

Although further studies are needed to determine a mechanism for the observed disparity in outcomes among highly aneuploid tumors treated with sequential versus concurrent radiotherapy and ICB, it is possible that this is driven in part by the previously described inherent resistance of highly aneuploid tumors to radiotherapy²⁸. Aneuploidy may also induce immune suppression through induction of proteotoxic stress due to an increase in gene products created by arm-level SCNAs as well as less effective neoantigen major histocompatibility complex binding and presentation. However, the addition of ICB to radiotherapy may partially overcome these immunosuppressive effects by improving antigen processing and presentation while recruiting novel CD8⁺ T cells into the tumor microenvironment. Concomitantly, immunogenic cell death due to SBRT could create damage-associated molecular patterns that can then be recognized by the novel T cell repertoire. Administration of ICB after radiotherapy as opposed to simultaneously may limit the beneficial effect of the addition of radiotherapy.

Despite the limited sample size of matched metastasis biopsies that passed the stringent quality standards, this is nevertheless a comparatively large biopsy-driven study of SBRT in NSCLC and examines the impact of radiotherapy versus radiotherapy+ICB. This allowed us to develop and test hypotheses related to tumor aneuploidy in two independent metastatic NSCLC cohorts. However, an important caveat of the analysis is that the on-treatment biopsy in the sequential arm does not reflect the post-ICB response. Thus, it is possible that the immediate immunosuppressive effects following SBRT can be attenuated by the sequential administration of ICB, as recently suggested³⁰. In addition, the limited quantity of tumor tissue, as well as the moderate genomic sequencing depth, constrained the ability to assess intratumoral heterogeneity of treatment-induced clonal evolution. A fraction of tumors in the study exhibited low tumor purity, which can affect copy number identification as well. Nevertheless, among highly aneuploid tumors, the inventors found improved survival with concurrent as opposed to sequential therapy, suggesting that the timing of radiotherapy and ICB, and the resulting immunogenomic consequences contribute to the survival differences among patients with metastatic NSCLC. The identification of aneuploidy as a biomarker of benefit of concurrent radiotherapy and ICB in two independent metastatic NSCLC cohorts suggests that this observation is reproducible.

Taken together, the results represent a comprehensive immunogenomic study of the effects of combination SBRT and ICB in metastatic NSCLC. The inventors demonstrate the immunogenic interaction of concurrent SBRT and ICB, and identify tumor aneuploidy as a potential biomarker of the response to combination radiotherapy and ICB. The findings suggest that existing clinical genomic sequencing architecture can inform strategies to optimize the integration of radiotherapy and ICB currently being tested in clinical trials of metastatic NSCLC and many other human cancers.

### Methods

### Patient Selection and Sample Collection

This study complies with all ethical regulations and was approved by the University of Chicago Biological Sciences Division Institutional Review Board Committee (IRB17-0547-CR003). Patients enrolled in the COSINR study¹⁵ (NCT03223155) were eligible for analysis. The COSINR study is a randomized phase I/II trial designed to evaluate the safety and efficacy of combination ICB using ipi/nivo plus sequential or concurrent SBRT as a first-line treatment for patients with stage IV NSCLC. Eligibility criteria include ECOG 0 or 1 performance status, measurable disease defined by Response Evaluation Criteria in Solid Tumours (RECIST) v1.1, no symptomatic central nervous system disease and no autoimmune or immunodeficiency syndromes. Without consideration of PD-L1 expression or TMB, patients were randomized to SBRT to two to four metastatic sites with concurrent or sequential (within 7 days) immunotherapy. A total of 37 patients were treated on the phase I component of the COSINR study. Matched tumor biopsies of a single irradiated metastasis were obtained prior to treatment and following completion of SBRT (sequential arm) or completion of SBRT and one cycle of ICB (concurrent arm). Biopsies were obtained approximately 3 to 4 weeks apart. PD-L1 expression was determined as the percentage of tumor cells expressing PD-L1 by clinical immunohistochemistry testing, as previously described¹⁵. Diagnostic imaging with positron emission tomography-computed tomography and whole-body computed tomography was performed with cycle 2 and computed tomography was at minimum performed with every other cycle thereafter until treatment completion or discontinuation (FIG. 3A). Further information on research design is available in the Nature Research Reporting Summary linked to this article.

Of 37 eligible patients, 22 had successful whole exome sequencing of pretreatment biopsies as well as matched peripheral blood; of these, on-treatment biopsies for 18 patients were included. Of the 18 patients, 15 underwent successful pretreatment and on-treatment RNA-seq and were included in the analysis (Consolidated Standards of Reporting Trials (CONSORT) diagram shown in FIG. 9B). Although additional fresh frozen tumor samples were unavailable as all of these samples were used for whole exome sequencing and RNA-seq, the inventors previously acquired independent FFPE core biopsies from each patient's irradiated metastasis at the specified time points, which allowed us to assess the concordance between the fresh frozen and FFPE biopsies. Manual review of pathologic and sequencing data (mutations and copy number profiles) was conducted to ensure that tumor variants were detected in all samples. The inventors identified two patients (12 and 13) who underwent biopsy and who had subsequent molecular analysis of a partially irradiated metastasis, which was permitted in the study. In both patients, the planning target volume covered 95-99% of the original internal target volume; therefore, the majority of the gross tumor received the full radiation prescription dose. As such, the inventors included patients 12 and 13 in the molecular analysis. Patients with diploid tumors or those with no mutations in pretreatment samples were excluded. In addition, on-treatment samples in which no tumor variants were identified were assigned diploid status (ploidy=2, purity=0). These samples were used only to demonstrate elimination of tumor variants and for analysis of local immune transcriptomic milieu if immune and stromal elements were detected on pathologic review.

Validation of the findings was performed using a 58 patient subset of 139 patients with NSCLC who underwent next-generation targeted genomic sequencing and were treated with ICB (59% anti-PD-1 or anti-PD-L1 and 41% ICB in combination) at the institution, as previously described²⁴. The inventors selected patients with available segmented copy number data and split those patients into three well-defined subsets for further analysis: (1) ICB alone (no radiation; patients who received radiotherapy to the brain (radiosurgery or whole-brain radiation therapy) were excluded, n=30); (2) sequential radiotherapy and ICB (interval between treatments of 7-60 days; n=14); and (3) concurrent radiotherapy and ICB (interval between treatments of 0 days; n=14). In the sequential and concurrent groups, patients were selected only if they had received radiotherapy to at least one extracranial disease site regardless of radiotherapy dose or target organ site.

Copy number segmentation files for 500 lung adenocarcinoma samples from the TCGA Pan Cancer Atlas dataset²⁵ were downloaded from cBioPortal³¹ for analysis of the distribution of aneuploidy scores in a large multi-institutional cohort. In addition, copy number and clinical data for a cohort of 350 NSCLCs treated with ICB (94% anti-PD-1 or anti-PD-L1 and 6% ICB in combination) at the MSKCC¹⁰ were downloaded from cBioPortal to examine the relationship between TMB, aneuploidy and survival in the setting of ICB.

### DNA and RNA Extraction and Sequencing

Tumor DNA and RNA samples were isolated from snap-frozen tissue biopsies using the Qiagen Allprep DNA/RNA Mini kit according to the manufacturer's instructions. Germline DNA samples were isolated from the whole blood using the Qiagen PAXgene Blood DNA kit according to the manufacturer's instructions. Tumor RNA samples were treated with DNase, and quality control was performed using the Agilent RNA 6000 Pico kit. Ribo-Zero total RNA libraries were prepared and sequenced on the Illumina NovaSeq 6000 system at a depth of approximately 60 million reads per sample. Tumor DNA and germline samples were treated with RNase, and quality control was performed using the Agilent High Sensitivity DNA kit and Agilent Genomic DNA Fragment Analyzer. SureSelect V7 libraries were then prepared and sequenced on the Illumina NovaSeq 6000 system.

### Whole Exome Sequencing Sample Processing and Variant Calling

Raw fastq files were first trimmed using Trimmomatic v0.39³². The trimmed reads were aligned to the hg38 human reference genome³³ using BWAmem v0.7.1³⁴ and sorted using samtools v1.11³⁵. PCR duplicates were identified using Picardtools v2.23.8 MarkDuplicates and further recalibrated using GATK³⁶ v4.1.9.0 BaseRecalibrator and ApplyBQSR with known indels from the GATK resource bundle in the Agilent sureSelect Human exome V7 bait set. Somatic variant calling for each tumor-normal pair was performed using GATK4 Mutect2. The called somatic variants were further filtered by GATK FilterMutectCalls using contamination estimates from CalculateContamination. The filtered VCF was annotated with ANNOVAR v2019102437 and GATK funcotator to generate a MAF file for each tumor sample. The variants underwent 8-oxoguanine (8-oxoG) filtering, as described below. Further filtering was performed wherein variants with a variant allele fraction of ≤0.1 or with ≤5 supporting variant reads were excluded to reduce the burden of sequencing artifacts. The inventors rescued variants that were listed as pathogenic or likely to be pathogenic in OncoKB (described below), were seen in COSMIC v91 (database downloaded on Jun. 30, 2021) at least three times or were called in the paired sample when both pretreatment and on-treatment samples were available. FACETS³⁸ v0.6 was used to call somatic CNAs and determine sample purity and ploidy using the default settings.

8-oxoG Artifact Filtering

Owing to the observed high burden of C>A transversions at low variant allele fractions, the inventors determined that it was necessary to perform additional filtering to remove 8-oxoG sequencing artifacts. The inventors performed this filtering, as previously described³⁹. In brief, C>A or G>T mutations were interrogated for a potential artifactual origin by computing the fraction of 8-oxoG (FoxoG) as the reads with G>T changes on read 1 and C>A changes on read 2 based on the strand-specific read counts as determined using MuTect2:

$$FoxoG = [ \begin{matrix}if C > A : F 2 R 1 / ( F 1 R 2 + F 2 R 1 ) \\ if G > T : F 1 R 2 / ( F 1 R 2 + F 2 R 1 )\end{matrix}$$

The inventors then removed all mutations where Tumor_LOD<−10+(100/3)×FoxoG.

### Determination of Pathogenicity of Genomic Alterations

Predicted mutation oncogenicity was retrieved from the OncoKB⁴⁰ application programming interface (v3.1) using the peptide change on Sep. 21, 2021. Mutations annotated as oncogenic or likely/predicted oncogenic were considered pathogenic. For gene-level copy number variants, the inventors included only those afflicting known oncogenes or tumor suppressors as defined in the OncoKB database. The inventors defined amplifications as loci (genes) called by FACETS as having a total copy number (TCN)≥(the average sample ploidy+3) and homozygous deletions as those with a TCN=0.

### CCF Calculation

Cancer cell fractions (CCFs) were obtained for each mutation using

previously described methods⁴¹. The expected variant allele fraction (VAF) was calculated for each potential CCF (C\hat{ }CF) between 0.001 and 1 in increments of 0.001 using the FACETS sample purity (p), allelic copy number (ACN) and TCN at the mutation locus.

$$E ( VAF ) = \frac{p \times \text{?} \times ACN}{2 \times ( 1 - p ) + p \times TCN}$$

The final CCF of the mutation was the candidate CCF that maximizes the binomial probability of observing the alternate (nₐₗₜ) and reference (n_{ref}) reads at the mutant locus.

$$CCF = \text{?} \vert arg max [ Binom ( {n}_{alt} \vert {n}_{ref} , E ( VAF ) ) ]$$

### TMB and Aneuploidy Score Calculation

TMB was defined as the number of nonsynonymous mutations in each sample divided by the sequencing bait size (COSINR, 35.7 Mb; UC, 2.8 Mb). Arm-level SCNAs were called using ASCETS²⁹ version 1.1 on the normalized copy number segmentation files from FACETS (COSINR) or CNVkit (UC), using a threshold for calling amplifications and deletions of +0.1. The aneuploidy score²³ for a sample was defined as the fraction of evaluable arms (ASCETS call of AMP, DEL, NEUTRAL or NC) afflicted by arm-level SCNAs (AMP or DEL). ASCETS was specifically designed and validated for use in targeted panel data and was therefore used in the UC cohort, as previously described²⁹.

### Mutational Signature Deconvolution

Mutational signature deconvolution was performed using the MutationalPatterns⁴² package (v4.1.1) in R on all samples with at least 20 mutations (n=32). After excluding mutations possibly to represent 8-oxoG artifacts, the inventors performed an initial fit of the COSMIC signatures to the refined mutation spectra in the sample using the fit_to_signatures_stricto function with a max_delta parameter of 0.02. The inventors observed examples of signature overfitting despite the strict refitting approach, with a multitude of mutational processes predicted to be active in each sample, including many that were not expected to be found in NSCLC. Therefore, the inventors refit the spectra using the list of only those signatures demonstrated to be found in lung adenocarcinoma or lung squamous tumors as previously described⁴³ and using information on the COSMIC website.

To confirm that the inventors had successfully removed artifactual mutations during 8-oxoG filtering, as described above, the inventors compared the mutation spectrum of the filtered mutations to the COSMIC signatures. As expected, the most similar COSMIC signature was SBS45 (oxidative damage by 8-oxoG) with cossim=0.96.

### RNA-Seq Pipeline

RNA-seq analysis was carried out by first aligning 100-bp-length paired-end reads to the human hg38 genome using the STAR⁴⁴ aligner v2.6.1d. The resulting BAM files were sorted by read name using samtools³⁵ v1.10. The total reads per gene were counted using htseq with the stranded option and using the Ensembl human hg38 list of coding exons for each gene as a reference. A matrix table of counts for each gene and sample was generated. The resulting table was analyzed for differential expression as paired samples (pretreatment versus on-treatment) using the R package DESeq2⁴⁵ using count values. Log₂(fold change) values were shrunk using the apeglm method⁴⁶.

### Single-Sample Gene Set Enrichment Analysis

ssGSEA was performed using the gsva package in R on the normalized RNA-seq FPKM (fragments per kilobase per million reads) matrix with the following parameters (method=‘ssgsea’, kcdf=‘Poisson’) on the set of hallmark gene sets (h.all.v7.4.symbols.gmt).

### Immune Signature Analysis

Cell-specific immune signatures were calculated using the xCell¹⁷ web portal (https://xcell.ucsf.edu/) using all signature matrices that included T cells (xCell, Bindea, Charoentong and Rooney) on the normalized expression (FPKM) matrix of all 30 paired RNA-seq samples. Effector T cell IFNγ¹⁴ and naive T cell signatures⁴⁷ were calculated using the R package singscore.

### HLA Typing and Neoantigen Binding Prediction

Computational human leukocyte antigen (HLA) typing was performed on each matched normal sample using PolySolver v4.0⁴⁸. For the neoantigen and neopeptide analyses, the inventors performed a modified version of the approach described in⁴⁹. Eight amino acids on either side of each nonsilent single-nucleotide variant with an annotated protein change were retrieved using the biomaRt⁵⁰ package, forming a 17-mer (17-base polymer) peptide. This peptide was transformed into 9-mer peptides using a sliding window approach. The inventors then used NetMHCpan⁵¹ v4.1 to predict the binding of each candidate neopeptide to the patient-specific HLA alleles. Neopeptides with a rank<2% were considered to be binders. The inventors then excluded any neopeptides that were not expressed in the harboring sample (FPKM=0). This enabled the analysis to at least partially capture the effects of upregulating and downregulating expression of neoantigens in on-treatment samples. Although an individual mutation could potentially create multiple neopeptides, in the analysis, neoantigens specifically refer to a subset of neopeptides in which each mutation can represent at most one neoantigen.

### TCR Clonotype Analysis

TCR clonotypes were determined from bulk RNA-seq using the MiXCR¹⁸ pipeline. The resulting clonotypes were used to calculate richness (the number of unique clonotypes in a sample) and evenness (the Shannon entropy of the clonotypes in a sample divided by the maximum possible entropy given the number of clonotypes (equal to log₂(richness))). Changes in TCR populations on therapy were categorized as follows: (1) novel (new clonotypes detected only in on-treatment sample); (2) elimination (clonotypes in pretreatment samples not detected on therapy); (3) expansion (existing clonotypes whose clone fraction increased by at least 10% on therapy); (4) contraction (existing clonotypes whose clone fraction was reduced by at least 10% on therapy); and (5) persistence (existing clonotypes whose clone fraction changed less than 10% on therapy).

### Treatment Response and Survival Analysis

RECIST v1.1⁵² was used to assess unirradiated tumor response. Irradiated tumors were not evaluated as part of the treatment response, consistent with RECIST principles. PFS was defined as the time from starting RT to progression or death, and OS as the time from starting RT to death. Patients were scored as censored at the time of the last follow-up. Survival analysis was performed using the survminer and survival packages, using the survdiff( ) function in R for Kaplan-Meier survival analyses. For univariable and multivariable Cox proportional hazards analysis, the coxpho function was used. Unadjusted or adjusted hazard ratios for survival were reported as appropriate. For analyses of specific genomic predictors of response, the inventors included as covariates TMB for mutations and aneuploidy score for CNAs to correct for background levels of genomic instability.

### Leave-One-Out Cross Validation

Leave-one-out cross validation was performed to determine an optimal threshold of high versus low aneuploidy. Candidate thresholds 0.02 to 0.60 in steps of 0.02 were analyzed. A Kaplan-Meier survival model of radiotherapy+ICB versus ICB alone was computed for OS of the patients with tumors harboring an aneuploidy score greater than or equal to each candidate threshold. This process was repeated n times, where n is the cohort size, leaving out one unique patient in each iteration. For each candidate threshold, the mean log-rank P value across the n subsets and 95% confidence intervals were computed as well as the mean difference in 12-month survival between the combination radiotherapy+ICB group and the ICB group. The optimal threshold was determined by selecting the candidate threshold at which the difference between the mean P value and 12-month survival difference was greatest.

### Multiplexed Immunofluorescence

Multiplexed immunofluorescence was successfully performed on pretreatment and on-treatment samples from 12 of 15 patients included in the RNA-seq analyses (Supplementary Table 2). A panel of six markers was developed to characterize the tumor microenvironment (Supplementary Table 20). The staining was performed using the Opal Polaris 7-color manual IHC detection kit (Akoya Bio, NEL861001KT) following the manufacturer's instructions. The FFPE tissue slides were deparaffinized by baking at 65° C. for 1 h, followed by incubating in xylenes three times for 10 min each. The tissue slides were then rehydrated in a series of ethanol gradients, fixed in 10% NBF solution and rinsed in distilled water. Next, antigen retrieval was performed using a pressure cooker at the low-pressure setting for 15 min in high-pH antigen retrieval buffer. The tissue slides were then blocked in tissue blocking buffer and incubated with the first primary antibody for 1 h at room temperature (20° C.) in a humidifying chamber. After a thorough wash with TBST buffer, the slides were incubated with secondary antibody-HRP buffer for 10 min at room temperature. The slides were washed again in TBST buffer and incubated with Opal fluorophores for 10 min at room temperature followed by TBST washes. The pressure cooker treatment was performed again to wash away the excess Opal fluorophore and retrieve antigens for the next primary antibody staining. The inventors repeated the above steps sequentially for each additional antibody. After the final antibody staining, the tissue slides were counterstained with DAPI for 5 min at room temperature, rinsed in water and mounted with ProLong.

Diamond Antifade Mountant (Life Technologies, P36961). After air drying, the multiplexed slides were scanned using an Akoya Vectra Polaris multispectral scanner. The regions of interest were selected using Phenochart software v1.1.0 (Akoya Bio) and image analysis, and tissue and cell segmentation as well as cell phenotype classification was performed using InForm (Akoya Bio) as described in the user manual. CD8⁺ T cells were defined as cells expressing both CD3 and CD8. Tumor cells were defined as cells expressing TTF1 and CK5. Cell densities were calculated by taking the total number of cells in each category across all selected regions of interest and dividing by the sum of the areas of each region.

### Statistics and Reproducibility

For enrichment analyses, the inventors included only genomic alterations that were present in at least three samples in the cohort or subgroup being analyzed (for example, within a treatment arm) to reduce the likelihood of type I error. When comparing continuous dependent variables, the inventors used a Mann-Whitney test or Kruskal-Wallis test, as appropriate. Splitting variables at the median was performed using the ntile function in R. When samples from the same patient were compared, the inventors used a paired test design. For comparisons of two categorical variables, the inventors used Fisher's exact test. For comparisons of two continuous variables, a Spearman correlation was used. Unless otherwise specified, all tests were performed in R v4.1.1 and were two-tailed. P<0.05 and false discovery rate (FDR)<0.1 were considered significant. Multiple comparison correction was performed using the Benjamini-Hochberg⁵³ method as implemented in the qvalue R package. No statistical method was used to predetermine sample sizes, but the sample sizes are similar to those reported in similar studies^{26,49}. Exclusion criteria are described above. Participants were randomly assigned to sequential or concurrent SBRT+ipi/nivo as described above. The investigators were not blinded during data collection, analysis, experiments or outcome assessment.

### Example 4: Aneuploidy Survival Differences

TABLE 1
high vs. low aneuploidy scores (split at the median
value within each tumor type) within each tumor type.

| | | | | Two year |
|---|---|---|---|---|
| | | Two year | Two year | survival: |
| | | survival: | survival: | high AS − |
| Cancer Type | n | high AS (%) | low AS (%) | low AS (%)* |
| Colorectal | 110 | 29.7 | 66.7 | −37.1 |
| Cancer | | | | |
| Breast Cancer | 44 | 0.0 | 25.4 | −25.4 |
| Cancer of | 88 | 29.0 | 50.3 | −21.3 |
| Unknown | | | | |
| Primary | | | | |
| Bladder Cancer | 215 | 34.1 | 46.8 | −12.6 |
| Renal Cell | 151 | 62.4 | 70.4 | −8.0 |
| Carcinoma | | | | |
| All patients | 1660 | 40.5 | 47.1 | −6.6 |
| Esophagogastric | 126 | 23.6 | 29.2 | −5.5 |
| Cancer | | | | |
| Non-Small Cell | 350 | 30.1 | 34.9 | −4.8 |
| Lung Cancer | | | | |
| Melanoma | 320 | 64.0 | 68.3 | −4.3 |
| Head and Neck | 139 | 34.9 | 36.1 | −1.2 |
| Cancer | | | | |
| Glioma | 117 | 20.4 | 9.0 | 11.4 |

*Difference in 2 year overall survival, which was obtained by taking high AS − low AS (negative values indicate worse overall survival for high aneuploidy).

Table 1: high vs. low aneuploidy scores (split at the median value within each tumor type) within each tumor type.

TABLE 2
high vs. low aneuploidy scores specifically for
tumors with low tumor mutational burden (TMB).

| | | Two year | Two year | Two year |
|---|---|---|---|---|
| | | survival: | survival: | survival: low |
| | | low TMB | low TMB | TMB high AS − |
| Cancer Type | n | high AS (%) | low AS (%) | low AS (%) |
| Cancer of | 88 | 13.3 | 53.8 | −40.5 |
| Unknown | | | | |
| Primary | | | | |
| Colorectal | 110 | 26.5 | 59.7 | −33.2 |
| Cancer | | | | |
| Breast Cancer | 44 | 0.0 | 27.9 | −27.9 |
| Bladder Cancer | 215 | 29.6 | 43.6 | −14.0 |
| Non-Small Cell | 350 | 21.6 | 35.2 | −13.6 |
| Lung Cancer | | | | |
| Renal Cell | 151 | 60.2 | 70.3 | −10.1 |
| Carcinoma | | | | |
| All patients | 1660 | 35.7 | 45.4 | −9.7 |
| Melanoma | 320 | 60.2 | 65.1 | −4.9 |
| Head and Neck | 139 | 25.8 | 29.5 | −3.7 |
| Cancer | | | | |
| Glioma | 117 | 18.6 | 12.5 | 6.1 |
| Esophagogastric | 126 | 21.0 | 14.7 | 6.3 |
| Cancer | | | | |

TABLE 3
median aneuploidy score by tumor type.

| Cancer Type | Median_as* |
|---|---|
| Bladder Cancer | 0.3414634 |
| Breast Cancer | 0.4634146 |
| Cancer of Unknown Primary | 0.4146341 |
| Colorectal Cancer | 0.2682927 |
| Esophagogastric Cancer | 0.3414634 |
| Glioma | 0.3414634 |
| Head and Neck Cancer | 0.2682927 |
| Melanoma | 0.4024390 |
| Non-Small Cell Lung Cancer | 0.3658537 |
| Renal Cell Carcinoma | 0.2926829 |

*High aneuploidy score (AS) is a value greater than or equal to the median AS value within each tumor type.

TABLE 4
Absolute 1-year overall survival by aneuploidy
group across treatment arms.

| | 1-year overall survival | | | |
|---|---|---|---|---|
| | | ICI + | ICI + RT | ICI + RT |
| Group | ICI alone | Any RT | (sequential) | (concurrent) |
| High aneuploidy | 30.8% | 59.2% | 38.1% | 76.2% |
| Low aneuploidy | 53.9% | 48.6% | 42.9% | 60.0% |

TABLE 5
Percent difference comparing ICI alone to the other
groups of ICI + RT within each aneuploidy group.

| | 1-year overall survival difference | | | |
|---|---|---|---|---|
| | | ICI + | ICI + RT | ICI + RT |
| Group | ICI alone | Any RT | (sequential) | (concurrent) |
| High aneuploidy | Reference | +28.4% | +7.3% | +45.4% |
| Low aneuploidy | Reference | −5.3% | −11.0% | +6.1% |

Tables 4 and 5 show high aneuploidy tumors (which includes metastatic NSCLC patients) have a large survival improvement with radiotherapy which may be driven by the patients receiving concurrent radiotherapy and immune checkpoint inhibitors. By contrast, the low aneuploid tumors relatively less survival difference with the use of radiotherapy.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

### REFERENCES FOR EXAMPLES 1 AND 2

1. Samstein, R. M. et al. Tumor mutational load predicts survival after immunotherapy across multiple cancer types. Nat. Genet. 51, 202-206 (2019).
2. Strickler, J. H., Hanks, B. A. & Khasraw, M. Tumor mutational burden as a predictor of immunotherapy response: is more always better?Clin. Cancer Res. 27, 1236 (2021).
3. Schumacher, T. N. & Schreiber, R. D. Neoantigens in cancer immunotherapy. Science 348, 69-74 (2015).
4. Hellmann, M. D. et al. Nivolumab plus ipilimumab in lung cancer with a high tumor mutational burden. N. Engl. J. Med. 378, 2093-2104 (2018).
5. Fehrenbacher, L. et al. Atezolizumab versus docetaxel for patients with previously treated non-small-cell lung cancer (POPLAR):
   a multicentre, open-label, phase 2 randomised controlled trial. Lancet 387, 1837-1846 (2016).
6. Romero, D. TMB is linked with prognosis. Nat. Rev. Clin. Oncol. 16, 336-336 (2019).
7. Rousseau, B. et al. The spectrum of benefit from checkpoint blockade in hypermutated tumors. N. Engl. J. Med. 384, 1168-1170 (2021).
8. Taylor, A. M. et al. Genomic and functional approaches to understanding cancer aneuploidy. Cancer Cell 33, 676-689.e3 (2018).
9. Lamberti, G. et al. Clinicopathological and genomic correlates of programmed cell death ligand 1 (PD-L1) expression in nonsquamous non-small-cell lung cancer. Ann. Oncol. 31, 807-814 (2020).
10. Davoli, T., Uno, H., Wooten, E. C. & Elledge, S. J. Tumor aneuploidy correlates with markers of immune evasion and with reduced response to immunotherapy. Science 355, eaaf8399 (2017).
11. Hieronymus, H. et al. Tumor copy number alteration burden is a pan-cancer prognostic factor associated with recurrence and death. eLife 7, e37294 (2018).
12. Ben-David, U. & Amon, A. Context is everything: aneuploidy in cancer. Nat. Rev. Genet. 21, 44-62 (2020).
13. Spurr, L. F. et al. Highly aneuploid non-small cell lung cancer shows enhanced responsiveness to concurrent radiation and immune checkpoint blockade. Nat. Cancer https://doi. org/10.1038/s43018-022-00467-x (2022).
14. Spurr, L. F. et al. Quantification of aneuploidy in targeted sequencing data using ASCETS. Bioinformatics 37, 2461-2463 (2020).
15. Cerami, E. et al. The cBio cancer genomics portal: an open platform for exploring multidimensional cancer genomics data. Cancer Discov. 2, 401-404 (2012).
16. Spurr, L. F. lfspurr/Aneuploidy-ICB: v1.0. Zenodo https://doi.org/10.5281/ZENODO.7181524 (2022).

### REFERENCES FOR EXAMPLE 3

1. Herbst, R. S., Morgensztern, D. & Boshoff, C. The biology and management of non-small cell lung cancer. Nature 553, 446-454 (2018).
2. Gandhi, S. J. et al. Awakening the immune system with radiation: optimal dose and fractionation. Cancer Lett. 368, 185-190 (2015).
3. Shabason, J. E. & Minn, A. J. Radiation and immune checkpoint blockade: from bench to clinic. Semin. Radiat. Oncol. 27, 289-298 (2017).
4. Twyman-Saint Victor, C. et al. Radiation and dual checkpoint blockade activate non-redundant immune mechanisms in cancer. Nature 520, 373-377 (2015).
5. Weichselbaum, R. R., Liang, H., Deng, L. & Fu, Y.-X. Radiotherapy and immunotherapy: a beneficial liaison?Nat. Rev. Clin. Oncol. 14, 365-379 (2017).
6. Chen, H.-Y. et al. Inhibiting the CD8⁺ T cell infiltration in the tumor microenvironment after radiotherapy is an important mechanism of radioresistance. Sci. Rep. 8, 11934 (2018).
7. Arina, A., Gutiontov, S. I. & Weichselbaum, R. R. Radiotherapy and immunotherapy for cancer: from ‘systemic’ to ‘multisite’. Clin. Cancer Res. 26, 2777 (2020).
8. Antonia, S. J. et al. Overall survival with durvalumab after chemoradiotherapy in stage III NSCLC. N. Engl. J. Med. 379, 2342-2350 (2018).
9. Kelly, R. J. et al. Adjuvant nivolumab in resected esophageal or gastroesophageal junction cancer. N. Engl. J. Med. 384, 1191-1203 (2021).
10. Samstein, R. M. et al. Tumor mutational load predicts survival after immunotherapy across multiple cancer types. Nat. Genet. 51, 202-206 (2019).
11. Strickler, J. H., Hanks, B. A. & Khasraw, M. Tumor mutational burden as a predictor of immunotherapy response: is more always better?Clin. Cancer Res. 27, 1236 (2021).
12. Schumacher Ton, N. & Schreiber Robert, D. Neoantigens in cancer immunotherapy. Science 348, 69-74 (2015).
13. Hellmann, M. D. et al. Nivolumab plus ipilimumab in lung cancer with a high tumor mutational burden. N. Engl. J Med. 378, 2093-2104 (2018).
14. Fehrenbacher, L. et al. Atezolizumab versus docetaxel for patients with previously treated non-small-cell lung cancer (POPLAR): a multicentre, open-label, phase 2 randomised controlled trial. Lancet 387, 1837-1846 (2016).
15. Bestvina, C. M. et al. A phase 1 trial of concurrent or sequential ipilimumab, nivolumab, and stereotactic body radiotherapy in patients with stage IV NSCLC study. J. Thorac. Oncol. 17, 130-140 (2022).
16. Paz-Ares, L. G. et al. First-line nivolumab plus ipilimumab in advanced nsclc: 4-year outcomes from the randomized, open-label, phase 3 CheckMate 227 Part 1 trial. J. Thorac. Oncol. 17, 289-308 (2022).
17. Aran, D., Hu, Z. & Butte, A. J. xCell: digitally portraying the tissue cellular heterogeneity landscape. Genome Biol. 18, 220 (2017).
18. Bolotin, D. A. et al. MiXCR: software for comprehensive adaptive immunity profiling. Nat. Methods 12, 380-381 (2015).
19. Reuben, A. et al. TCR repertoire intratumor heterogeneity in localized lung adenocarcinomas: an association with predicted neoantigen heterogeneity and postsurgical recurrence. Cancer Discov. 7, 1088 (2017).
20. Joshi, K. et al. Spatial heterogeneity of the T cell receptor repertoire reflects the mutational landscape in lung cancer. Nat. Med. 25, 1549-1559 (2019).
21. Lamberti, G. et al. Clinicopathological and genomic correlates of programmed cell death ligand 1 (PD-L1) expression in nonsquamous non-small-cell lung cancer. Ann. Oncol. 31, 807-814 (2020).
22. Davoli, T., Uno, H., Wooten, E. C. & Elledge, S. J. Tumor aneuploidy correlates with markers of immune evasion and with reduced response to immunotherapy. Science 355, eaaf8399 (2017).
23. Taylor, A. M. et al. Genomic and functional approaches to understanding cancer aneuploidy. Cancer Cell 33, 676-689.e3 (2018).
24. Gutiontov, S. I. et al. CDKN2A loss-of-function predicts immunotherapy resistance in non-small cell lung cancer. Sci. Rep. 11, 20059 (2021).
25. Hoadley, K. A. et al. Cell-of-origin patterns dominate the molecular classification of 10,000 tumors from 33 types of cancer. Cell 173, 291-304.e6 (2018).
26. Formenti, S. C. et al. Radiotherapy induces responses of lung cancer to CTLA-4 blockade. Nat. Med. 24, 1845-1851 (2018).
27. Golden, E. B. & Apetoh, L. Radiotherapy and immunogenic cell death. Semin. Radiat. Oncol. 25, 11-17 (2015).
28. Jia, Q. et al. Mutational burden and chromosomal aneuploidy synergistically predict survival from radiotherapy in non-small cell lung cancer. Commun. Biol. 4, 131 (2021).
29. Spurr, L. F. et al. Quantification of aneuploidy in targeted sequencing data using ASCETS. Bioinformatics 37, 2461-2463 (2021).
30. Wei, J. et al. Sequence of αPD-1 relative to local tumor irradiation determines the induction of abscopal antitumor immune responses. Sci. Immunol. 6, eabg0117 (2021).
31. Cerami, E. et al. The cBio cancer genomics portal: an open platform for exploring multidimensional cancer genomics data. Cancer Discov. 2, 401-404 (2012).
32. Bolger, A. M., Lohse, M. & Usadel, B. Trimmomatic: a flexible trimmer for Illumina sequence data. Bioinformatics 30, 2114-2120 (2014).
33. Schneider, V. A. et al. Evaluation of GRCh38 and de novo haploid genome assemblies demonstrates the enduring quality of the reference assembly. Genome Res. 27, 849-864 (2017).
34. Li, H. & Durbin, R. Fast and accurate short read alignment with Burrows-Wheeler transform. Bioinformatics 25, 1754-1760 (2009).
35. Li, H. et al. The sequence alignment/map format and saMtools. Bioinformatics 25, 2078-2079 (2009).
36. McKenna, A. et al. The Genome Analysis Toolkit: a MapReduce framework for analyzing next-generation DNA sequencing data. Genome Res. 20, 1297-1303 (2010).
37. Ellrott, K. et al. Scalable open science approach for mutation calling of tumor exomes using multiple genomic pipelines. Cell Syst. 6, 271-281.e7 (2018).
38. Shen, R. & Seshan, V. E. FACETS: allele-specific copy number and clonal heterogeneity analysis tool for high-throughput DNA sequencing. Nucleic Acids Res. 44, e131 (2016).
39. Costello, M. et al. Discovery and characterization of artifactual mutations in deep coverage targeted capture sequencing data due to oxidative DNA damage during sample preparation. Nucleic Acids Res. 41, e67 (2013).
40. Chakravarty, D. et al. OncoKB: a precision oncology knowledge base. JCO Precis. Oncol. https://doi.org/10.1200/PO.17.00011 (2017).
41. McGranahan, N. et al. Clonal status of actionable driver events and the timing of mutational processes in cancer evolution. Sci. Transl. Med. 7, 283ra54 (2015).
42. Blokzijl, F., Janssen, R., van Boxtel, R. & Cuppen, E. MutationalPatterns: comprehensive genome-wide analysis of mutational processes. Genome Med. 10, 33 (2018).
43. Alexandrov, L. B. et al. The repertoire of mutational signatures in human cancer. Nature 578, 94-101 (2020).
44. Dobin, A. et al. STAR: ultrafast universal RNA-seq aligner. Bioinformatics 29, 15-21 (2013).
45. Love, M. I., Huber, W. & Anders, S. Moderated estimation of fold change and dispersion for RNA-seq data with DESeq2. Genome Biol. 15, 550 (2014).
46. Zhu, A., Ibrahim, J. G. & Love, M. I. Heavy-tailed prior distributions for sequence count data: removing the noise and preserving large differences. Bioinformatics 35, 2084-2092 (2019).
47. Szabo, P. A. et al. Single-cell transcriptomics of human T cells reveals tissue and activation signatures in health and disease. Nat. Commun. 10, 4706 (2019).
48. Shukla, S. A. et al. Comprehensive analysis of cancer-associated somatic mutations in class I HLA genes. Nat. Biotechnol. 33, 1152-1158 (2015).
49. Riaz, N. et al. Tumor and microenvironment evolution during immunotherapy with nivolumab. Cell 171, 934-949.e16 (2017).
50. Durinck, S., Spellman, P. T., Birney, E. & Huber, W. Mapping identifiers for the integration of genomic datasets with the R/ Bioconductor package biomaRt. Nat. Protoc. 4, 1184-1191 (2009).
51. Reynisson, B., Alvarez, B., Paul, S., Peters, B. & Nielsen, M. NetMHCpan-4.1 and NetMHCIIpan-4.0: improved predictions of MHC antigen presentation by concurrent motif deconvolution and integration of MS MHC eluted ligand data. Nucleic Acids Res. 48, W449-W454 (2020).
52. Eisenhauer, E. A. et al. New response evaluation criteria in solid tumours: revised RECIST guideline (version 1.1). Eur. J. Cancer 45, 228-247 (2009).
53. Benjamini, Y. & Hochberg, Y. Controlling the false discovery rate: a practical and powerful approach to multiple testing. J. Roy. Stat. Soc. B 57, 289-300 (1995).

What is claimed is:

1. A method of treating a cancer in a patient, the method comprising administering to the patient radiotherapy and/or an immune checkpoint blockade (ICB) therapy, after the cancer is determined to have a high aneuploidy score.

2. The method of claim 1, wherein the cancer has an indication for radiotherapy administration.

3. The method of claim 1 or 2, wherein the cancer originated in an organ of the individual selected from the group consisting of bladder, blood, bone, bone marrow, brain, breast, colon, esophagus, duodenum, small intestine, large intestine, colon, rectum, anus, gum, a head organ, kidney, liver, lung, nasopharynx, a neck organ, ovary, pancreas, prostate, skin, stomach, testis, tongue, uterus, lymph, lymph node, muscle, fat, fibrous tissue, blood vessels and a combination thereof.

4. The method of any one of claims 1-3, wherein the cancer is a Stage I cancer, a Stage II cancer, a Stage III cancer, or a Stage IV cancer.

5. The method of any one of claims 1-4, wherein the cancer is a not a glioma.

6. The method of any one of claims 1-5, wherein the cancer comprises a cancer derived from endoderm tissue.

7. The method of any one of claims 1-6, wherein the cancer comprises a non small-cell lung cancer or a large cell carcinoma.

8. The method of any one of claims 1-4, wherein the cancer comprises a myeloma or a melanoma.

9. The method of any one of claims 1-8, wherein the cancer comprises an immunologically cold tumor.

10. The method of any one of claims 1-9, wherein the cancer is metastatic.

11. The method of any one of claims 1-10, wherein the ICB comprises an anti-PD-1 agent, an anti-PD-L1 agent, and/or an anti-CTLA-4 agent.

12. The method of claim 11, wherein the anti-PD-1 agent, an anti-PD-L1 agent, and/or an anti-CTLA-4 agent comprise an antibody, a small molecule, a biologic, an antisense oligonucleotide, and/or an RNAi molecule.

13. The method of claim 11 or 12, wherein the ICB comprises ipilimumab, nivolumab, pembrolizumab, atezolizumab, avelumab, durvalumab, cemiplimab, or spartalizumab, or any combination thereof.

14. The method of any one of claims 1-13, wherein the aneuploidy score is measured by arm-level somatic copy number alterations.

15. The method of any one of claims 1-14, wherein the aneuploidy score is measured by Arm-level Somatic Copy-number Events in Targeted Sequencing (ASCETS).

16. The method of any one of claims 1-15, wherein the cancer is determined to have a high aneuploidy score via biopsy and/or tumor resection.

17. The method of any one of claims 1-16, wherein the cancer is determined to have a high aneuploidy score by sequencing.

18. The method of any one of claims 1-17, wherein a high aneuploidy score comprises an aneuploidy score greater than a reference score.

19. The method of claim 18, wherein the reference score comprises an average aneuploidy score of a cohort of individuals.

20. The method of claim 19, wherein the cohort of individuals comprises individuals known to or diagnosed to have cancer of the same type as the cancer in the patient.

21. The method of any of claims 1-20, wherein the patient is administered radiotherapy and ICB therapy.

22. The method of any one of claims 1-21, wherein the patient receives radiotherapy while undergoing ICB therapy.

23. The method of any one of claims 1-21, wherein the radiotherapy and ICB therapy are administered sequentially.

24. The method of claim 23, wherein 1, 2, 3, 4, 5, 6, 7, 8, 9, 10, 11, 12, 13, or 14 days are between the radiotherapy and ICB therapy.

25. The method of claim 23, wherein the radiotherapy is administered 1, 2, 3, 4, 5, 6, 7 days and/or 1, 2, 3, or 4 weeks prior to administering the ICB therapy.

26. The method of claim 23, wherein the radiotherapy is administered 1, 2, 3, 4, 5, 6, 7 days and/or 1, 2, 3, or 4 weeks after administering the ICB therapy.

27. The method of any one of claims 1-26, wherein the patient is determined to have low tumor mutational burden.

28. The method of any one of claims 1-27, wherein the patient is determined to have high mutational tumor burden.

29. A method for determining the effectiveness of a radiotherapy and/or immune checkpoint blockade (ICB) therapy in treating a cancer in an individual, the method comprising determining an aneuploidy score of the cancer.

30. The method of claim 29, wherein the cancer has an indication for radiotherapy administration.

31. The method of claim 29 or 30, wherein the cancer originated in an organ of the individual selected from the group consisting of bladder, blood, bone, bone marrow, brain, breast, colon, esophagus, duodenum, small intestine, large intestine, colon, rectum, anus, gum, a head organ, kidney, liver, lung, nasopharynx, a neck organ, ovary, pancreas, prostate, skin, stomach, testis, tongue, uterus, lymph, lymph node, muscle, fat, fibrous tissue, blood vessels and a combination thereof.

32. The method of any one of claims 29-31, wherein the cancer is a Stage I cancer, a Stage II cancer, a Stage III cancer, or a Stage IV cancer.

33. The method of any one of claims 29-32, wherein the cancer comprises a non small-cell lung cancer or a large cell carcinoma.

34. The method of any one of claims 29-32, wherein the cancer comprises a myeloma or a melanoma.

35. The method of any one of claims 29-34, wherein the cancer comprises an immunologically cold tumor.

36. The method of any one of claims 29-35, wherein the cancer is metastatic.

37. The method of any one of claims 29-36, wherein the ICB therapy comprises an anti-PD-1 agent, an anti-PD-L1 agent, and/or an anti-CTLA-4 agent.

38. The method of claim 37, wherein the anti-PD-1 agent, an anti-PD-L1 agent, and/or an anti-CTLA-4 agent comprise an antibody, a small molecule, a biologic, an antisense oligonucleotide, and/or an RNAi molecule.

39. The method of claim 37 or 38, wherein the ICB comprises ipilimumab, nivolumab, pembrolizumab, atezolizumab, avelumab, durvalumab, cemiplimab, or spartalizumab, or any combination thereof.

40. The method of any one of claims 29-39, wherein the aneuploidy score is measured by arm-level somatic copy number alterations.

41. The method of any one of claims 29-40, wherein the aneuploidy score is measured by Arm-level Somatic Copy-number Events in Targeted Sequencing (ASCETS).

42. The method of any one of claims 29-41, wherein the aneuploidy score is measured via biopsy and/or tumor resection.

43. The method of any one of claims 29-42, wherein the aneuploidy score is measured by sequencing.

44. The method of any one of claims 29-43, wherein a high aneuploidy score comprises an aneuploidy score greater than a reference score.

45. The method of claim 44, wherein the reference score comprises an average aneuploidy score of a cohort of individuals.

46. The method of claim 45, wherein the cohort of individuals comprises individuals known to or diagnosed to have cancer of the same type as the cancer in the patient.

47. The method of any one of claims 29-46, further comprising administering to the individual a radiotherapy and/or ICB therapy.

48. The method of any one of claims 29-47, wherein the individual has received a radiotherapy and/or ICB therapy.

49. The method of any one of claims 29-48, further comprising measuring tumor mutational burden.

50. The method of claim 49, wherein the individual is determined to have low tumor mutational burden.

51. The method of claim 49, wherein the individual is determined to have high tumor mutational burden.

52. A method of treating cancer in a patient that has received radiotherapy, the method comprising administering an immune checkpoint blockade (ICB) therapy if the patient is determined to have an increase in at least one immune checkpoint gene product after receiving the radiotherapy.

53. The method of claim 52, wherein the ICB therapy comprises an anti-PD-1 agent, an anti-PD-L1 agent, and/or an anti-CTLA-4 agent.

54. The method of claim 53, wherein the anti-PD-1 agent, an anti-PD-L1 agent, and/or an anti-CTLA-4 agent comprise an antibody, a small molecule, a biologic, an antisense oligonucleotide, and/or an RNAi molecule.

55. The method of claim 53 or 54, wherein the ICB therapy comprises ipilimumab, nivolumab, pembrolizumab, atezolizumab, avelumab, durvalumab, cemiplimab, or spartalizumab, or any combination thereof.

56. The method of any one of claims 52-55, wherein the cancer is metastatic.

57. The method of any one of claims 29-56, wherein the cancer is Stage I, Stage II, Stage III, or Stage IV.

58. The method of any one of claims 52-53xi, wherein the immune checkpoint gene product comprises mRNA and/or protein produced from a PD-1, PD-L1, and/or CTLA-4 gene.

59. The method of any one of claims 52-58, wherein the increase is determined relative to an amount of immune checkpoint gene product measured prior to the patient receiving the radiotherapy.

60. The method of any one of claims 52-58, wherein the increase is determined relative to a standard level of the immune checkpoint gene product in healthy individuals.

61. The method of any one of claims 52-60, wherein the immune checkpoint gene product is measured in a biopsy.

62. The method of any one of claims 52-61, wherein the immune checkpoint gene product is measured in circulating immune cells.

63. The method of any one of claims 52-62, wherein the cancer has an indication for radiotherapy administration.

64. The method of any one of claims 52-63, wherein the cancer originated in an organ of the individual selected from the group consisting of bladder, blood, bone, bone marrow, brain, breast, colon, esophagus, duodenum, small intestine, large intestine, colon, rectum, anus, gum, a head organ, kidney, liver, lung, nasopharynx, a neck organ, ovary, pancreas, prostate, skin, stomach, testis, tongue, uterus, lymph, lymph node, muscle, fat, fibrous tissue, blood vessels and a combination thereof.

65. The method of any one of claims 52-64, wherein the cancer comprises a non small-cell lung cancer or a large cell carcinoma.

66. The method of any one of claims 52-64, wherein the cancer comprises a myeloma or a melanoma.

67. The method of any one of claims 52-66, wherein the cancer comprises an immunologically cold tumor.

68. A method of treating cancer in a patient, the method comprising administering to the patient a second therapy after the cancer is measured for an aneuploidy score and/or tumor mutational burden, wherein the patient has received a first therapy and wherein the second therapy comprises radiotherapy and/or an immune checkpoint blockade (ICB) therapy.

69. The method of claim 68, wherein the aneuploidy score and/or tumor mutational burden is measured before the patient has received the first therapy.

70. The method of claim 68 or 69, wherein the aneuploidy score and/or tumor mutational burden is measured during the patient receiving the first therapy.

71. The method of any one of claims 68-70, wherein the aneuploidy score and/or tumor mutational burden is measured after the patient has received the first therapy.

72. The method of any one of claims 68-71, wherein the aneuploidy score is a high aneuploidy score.

73. The method of claim 72, wherein the administering the second therapy comprises administering radiotherapy and an ICB.

74. The method of any one of claims 68-71, wherein the aneuploidy score is a low aneuploidy score.

75. The method of claim 74, wherein the administering the second therapy comprises administering an ICB.

76. The method of any one of claims 68-75, wherein the first therapy comprises an ICB.

77. The method of any one of claims 68-75, wherein the first therapy comprises radiotherapy and an ICB.

78. A method of improving an immune checkpoint blockade therapy received by a patient, the method comprising administering a radiotherapy to the patient, wherein the patient has been determined to have a high aneuploidy score and low tumor mutational burden.

79. A method of improving an immune checkpoint blockade therapy received by a patient, the method comprising administering a radiotherapy to the patient, wherein the patient has been determined to have a high aneuploidy score and/or low tumor mutational burden.
